(19) United States
(12) Patent Application Publication
(10) Pub. No.: US 2025/0054596 A1
(43) Pub. Date: Feb. 13, 2025

(54) COMMUNICATION SYSTEM FOR PATIENT SUPPORT APPARATUSES AND TEMPERATURE MANAGEMENT DEVICES

(71) Applicant: Stryker Corporation, Kalamazoo, MI (US)

(72) Inventors: Jerald A. Trepanier, Augusta, MI (US); Marco Constant, Portage, MI (US); Krishna Sandeep Bhimavarapu, Kalamazoo, MI (US); Kirby M. Neihouser, Portage, MI (US); Madhu Sandeep Thota, Portage, MI (US); Celso Henrique Farnese Pires Pereira, Portage, MI (US); Madhu Thomas, London, Ontario (CA)

(21) Appl. No.: 18/724,068

(22) PCT Filed: Feb. 23, 2023
(86) PCT No.: PCT/US2023/013722

§ 371 Date: Jun. 25, 2024

Related U.S. Application Data
(60) Provisional application No. 63/314,221, filed on Feb. 25, 2022.
(60) Provisional application No. 63/349,369, filed on Jun. 6, 2022.
(60) Provisional application No. 63/428,075, filed on Nov. 27, 2022.
(60) Provisional application No. 63/428,076, filed on Nov. 27, 2022.
(60) Provisional application No. 63/428,077, filed on Nov. 27, 2022.
(60) Provisional application No. 63/352,061, filed on Jun. 14, 2022.
(60) Provisional application No. 63/356,061, filed on Jun. 28, 2022.
(60) Provisional application No. 63/356,238, filed on Jun. 28, 2022.
(60) Provisional application No. 63/356,065, filed on Jun. 28, 2022.

(51) Int. Cl. G16H 20/17; A61M 37/00

(52) U.S. Cl. CPC G16H 20/17; A61M 37/00; A61M 2205/3368

(57) ABSTRACT

A patient support apparatus for supporting a patient includes first, second, and third transceivers mounted at first, second, and third locations, respectively. A controller is adapted to use radio frequency communication between the first, second, and third transceivers and a first device—such as a patient temperature management device—to determine a position of the first device relative to the patient support apparatus. The controller is further adapted to use data from one or more other devices to determine whether or not to associate the first device with the patient support apparatus. In some aspects, the controller uses the position and/or identity of one or more thermal pads and/or hoses to determine if the first device is to be associated with the patient support apparatus. Such association refers to the conclusion that the patient support apparatus and the first device are both being used with the same patient.

### CROSS-REFERENCE TO RELATED APPLICATIONS

This application claims priority to each of the following U.S. provisional patent applications: Ser. No. 63/314,221 filed Feb. 25, 2022, by inventors Jerald Trepanier et al. and entitled COMMUNICATION SYSTEM FOR PATIENT SUPPORT APPARATUSES AND TEMPERATURE MANAGEMENT DEVICES; Ser. No. 63/352,061 filed Jun. 14, 2022, by inventors Jerald Trepanier et al. and entitled COMMUNICATION SYSTEM FOR PATIENT SUPPORT APPARATUSES; Ser. No. 63/349,369 file Jun. 6, 2022, by inventors Krishna Sandeep Bhimavarapu et al. and entitled COMMUNICATION SYSTEM FOR PATIENT SUPPORT APPARATUSES; Ser. No. 63/356,061 filed Jun. 28, 2022, by inventors Krishna Sandeep Bhimavarapu et al. and entitled BADGE AND PATIENT SUPPORT APPARATUS COMMUNICATION SYSTEM; Ser. No. 63/356,065 filed Jun. 28, 2022, by inventors Jerald Trepanier et al. and entitled BADGE AND PATIENT SUPPORT APPARATUS COMMUNICATION SYSTEM; Ser. No. 63/356,238 filed Jun. 28, 2022, by inventors Madhu Sandeep Thota et al. and entitled BADGE AND PATIENT SUPPORT APPARATUS COMMUNICATION SYSTEM; Ser. No. 63/428,075 filed Nov. 27, 2022, by inventors Kirby Neihouser et al. and entitled COMMUNICATION TOOL FOR UWB-EQUIPPED PATIENT DEVICES; Ser. No. 63/428,076 filed Nov. 27, 2022, by inventors Celso Henrique Farnese Pires Pereira et al. and entitled COMMUNICATION SYSTEM FOR PATIENT SUPPORT APPARATUSES AND OTHER EQUIPMENT; and Ser. No. 63/428,077 filed Nov. 27, 2022, by inventors Madhu Sandeep Thota et al. and entitled PATIENT SUPPORT APPARATUS COMMUNICATION SYSTEM; the complete disclosures of all of which are incorporated herein by reference.

### BACKGROUND

The present disclosure relates to patient support apparatuses, such as beds, cots, stretchers, recliners, or the like. More specifically, the present disclosure relates to patient support apparatuses that communicate with one or more devices that are separate from the patient support apparatus itself.

Patients in hospitals often have one or more medical devices that are used in the care and treatment of the patient. These medical devices often generate data that is desirably displayed and/or recorded in the medical records of the particular patient with whom they are being used. In order for the data from these devices to be recorded in the correct patient's electronic medical record, a caregiver typically has to take one or more manual steps to associate these devices with a particular patient. In addition, if the caregiver wishes to see any of this data displayed on a display device associated with that particular patient, the caregiver has to take one or more manual steps to associate each medical device with the display device on which they wish the data to be displayed. Such display device association may require the caregiver to plug a cable from the medical device into a selected display device and/or take other steps to view the data from the medical device on the selected display device.

### SUMMARY

According to the various aspects described herein, the present disclosure is directed to a patient support apparatus that reduces the labor associated with reading, recording, and/or displaying medical device data. In some aspects, the patient support apparatus automatically detects the relative position of a medical device, such as a patient temperature management device, to the patient support apparatus and automatically associates the data from that particular medical device with the patient assigned to that patient support apparatus if the relative position meets one or more criteria. Alternatively, or additionally, the patient support apparatus automatically detects the relative positions of a main medical device and one or more ancillary devices relative to the patient support apparatus, and uses the relative positions of both the main device and the ancillary device(s) to determine whether the main medical device should be associated with the patient support apparatus or not. In some aspects, in response to associating the medical device to the patient support apparatus, the patient support apparatus automatically displays data from the device on a display associated with that particular patient and/or automatically sends data from the device to a server that automatically records the data in that particular patient's electronic medical records. Such automatic association, display, and/or recordation reduces the time and effort otherwise required of the caregiver to perform these tasks. These and other improvements and/or advantages over prior art patient support apparatuses and communication systems will become apparent in light of the following written description and the accompanying drawings.

According to a first aspect of the present disclosure, a patient support apparatus is provided that includes a support surface; a first transceiver coupled to a first location on the patient support apparatus; a second transceiver coupled to a second location on the patient support apparatus; a third transceiver coupled to a third location on the patient support apparatus; and a controller. The controller is adapted to use radio frequency (RF) communication between the first, second, and third transceivers and first and second devices to determine a first position of the first device relative to the patient support apparatus and a second position of a second device relative to the patient support apparatus. The controller is further adapted to use both the first position and the second position to determine whether or not to associate the first device with the patient support apparatus.

According to other aspects of the present disclosure, the controller is further adapted to perform a first comparison of the first position to a first volume of space and to perform a second comparison of the second position to a second volume of space, and to use both the first and second comparisons to determine whether or not to associate the first device with the patient support apparatus.

In some aspects, the controller is further adapted to associate the first device with the patient support apparatus if the first device is positioned inside of a first volume of space and the second device is positioned inside of a second volume of space different from the first volume of space. In such aspects, the controller is also adapted to not associate the first device with the patient support apparatus if any of the following are true: (a) the first device is positioned outside of the first volume of space, (b) the second device is positioned outside of the second volume of space, or (c) both the first device is positioned outside of the first volume of space and the second device is positioned outside of the second volume of space.

In some aspects, the first device is a temperature management device adapted to deliver temperature controlled fluid to the patient, and the second device is a thermal pad adapted to wrap around a portion of the patient and receive the temperature controlled fluid from the temperature management device.

The first volume of space is larger than the second volume of space in some aspects.

Both the first and second volumes of space are defined in a fixed manner with respect to the patient support apparatus in some aspects.

The patient support apparatus, in some aspects, further includes a network transceiver adapted to communicate with a server hosted on a computer network.

The controller, in some aspects, is further adapted to receive a first unique ID from the first device and, in response to associating the first device with the patient support apparatus, to transmit the first unique ID and a unique identifier of the patient support apparatus to the server.

The patient support apparatus, in some aspects, further includes a memory in which a first definition of the first volume of space is stored and the controller is further adapted to receive a first ID from the first device and to use the first ID to select the first volume of space for use in the first comparison.

In some aspects, the second definition of the second volume of space is also stored in the memory and the controller is further adapted to receive a second ID from the second device and to use the second ID to select the second volume of space for use in the second comparison.

The patient support apparatus, in some aspects, further includes a memory in which a plurality of space volumes are defined, and the controller is adapted to select a first volume of space volume from the plurality of space volumes based on a first ID received from the first device and to select a second volume of space from the plurality of space volumes based on a second ID received from the second device.

In some aspects, the controller is further adapted to use radio frequency (RF) communication between the first, second, and third transceivers and a third device to determine a third position of the third device relative to the patient support apparatus, and to use only the third position and not the second position to determine whether to associate the third device with the patient support apparatus or not.

The controller, in some aspects, is further adapted to associate the third device with the patient support apparatus if the third device is positioned inside of the first volume of space, regardless of the first position of the first device or the second position of the second device relative to the first or second volumes of space.

In some aspects, the controller is further adapted to also use the third position of the third device to determine whether to associate the first device with the patient support apparatus or not.

In some aspects, the first device is a temperature management device adapted to deliver temperature controlled fluid to the patient, the second device is a first tag attached to a first end of a hose fluidly coupled to the temperature management device, and the third device is a second tag attached to a second end of the hose. Alternatively, the third device may be a hose fluidly coupled to the temperature management device and the thermal pad.

In some aspects, the controller is further adapted to use radio frequency (RF) communication between the first, second, and third transceivers to determine a third position of a third device relative to the patient support apparatus and a fourth position of a fourth device relative to the patient support apparatus. The controller is further adapted to use both the third position and the fourth position to determine whether or not to associate the first device with the patient support apparatus.

In some aspects, the first device is a temperature management device adapted to deliver temperature controlled fluid to the patient, the second device is a thermal pad adapted to wrap around a portion of the patient and receive the temperature controlled fluid from the temperature management device, the third device is a first tag attached to a first end of a hose fluidly coupled to the temperature management device and the thermal pad, and the fourth device is a second tag attached to a second end of the hose.

In some aspects, the first transceiver, the second transceiver, and the third transceiver are all ultra-wideband transceivers.

The patient support apparatus, in some aspects, further includes a memory in which the first location, second location, and third location of the first, second, and third transceivers, respectively, is stored. The controller is adapted to use the stored locations of the first, second, and third transceivers to determine the first and second positions.

In some aspects, the controller is adapted to use radio frequency (RF) communication between the first, second, and third transceivers and a fixed locator to determine a position of the fixed locator relative to the patient support apparatus, and the controller is further adapted to use only the position of the fixed locator to determine whether or not to associate the fixed locator with the patient support apparatus.

The patient support apparatus, in some aspects, further includes a microphone positioned onboard the patient support apparatus. The microphone is adapted to convert sounds of the patient's voice to audio signals, and the controller is adapted to transmit the audio signals to the fixed locator if the controller has associated the fixed locator with the patient support apparatus, and to not transmit the audio signals to the fixed locator if the controller has not associated the fixed locator with the patient support apparatus.

In some aspects, the patient support apparatus further includes a network transceiver adapted to communicate with a server of a local area network of a healthcare facility, and the controller is adapted to receive a location ID from the fixed locator, to send the location ID to the server using the network transceiver, and to thereafter receive a room number from the server via the network transceiver. The room number corresponds to a room in which the patient support apparatus is currently located.

The fixed locator, in some aspects, is adapted to be mounted at a fixed location within a room of a healthcare facility and to transmit a location ID to the patient support apparatus.

The first and second volumes of space, in some aspects, are defined with respect to the patient support apparatus and move as the patient support apparatus moves.

In some aspects, in response to the controller associating the first device with the patient support apparatus, the controller sends a message to an off-board server indicating that the first device and the patient support apparatus are being used with a common patient.

According to another aspect of the present disclosure, a patient support apparatus is provided that includes a support surface; a first transceiver coupled to a first location on the patient support apparatus; a second transceiver coupled to a second location on the patient support apparatus; a third transceiver coupled to a third location on the patient support apparatus; and a controller. The controller is adapted to use radio frequency (RF) communication between the first, second, and third transceivers and a first device to determine a first position of the first device relative to the patient support apparatus. The controller is further adapted to receive a first ID from the first device, to use the first ID to determine a first association condition, and to use the first position and the first association condition to determine whether or not to associate the first device with the patient support apparatus.

According to other aspects of the present disclosure, the first association condition defines a first volume of space inside which the first device must be positioned in order for the controller to associate the first device with the patient support apparatus.

In some aspects, the first association condition further defines a second volume of space inside of which a second device must be positioned in order for the controller to associate the first device with the patient support apparatus.

The first association condition, in some aspects, further defines a second ID of the second device.

The first association condition, in some aspects, further defines a first spatial condition between the first device and a third device that must be satisfied in order for the controller to associate the first device with the patient support apparatus, and a second spatial condition between the second device and a fourth device that also must be satisfied in order for the controller to associate the first device with the patient support apparatus.

The first spatial condition, in some aspects, defines a first threshold distance between the first device and the third device, and the second spatial condition, in some aspects, defines a second threshold distance between the second device and the fourth device.

The controller, in some aspects, is further adapted to receive association data from at least one of the third and fourth devices, and the association data indicates to the controller that the third device and fourth device are attached to the same hose.

The first association condition, in some aspects, is stored in a memory onboard the patient support apparatus.

The first association condition, in other aspects, is transmitted from the first device to the patient support apparatus.

In some aspects, the patient support apparatus further includes a network transceiver adapted to communicate with a server hosted on a computer network, and the controller is further adapted to receive a first unique ID from the first device. In response to associating the first device with the patient support apparatus, the controller is adapted to transmit the first unique ID and a unique identifier of the patient support apparatus to the server.

The controller, in some aspects, is further adapted to use radio frequency (RF) communication between the first, second, and third transceivers to determine a third position of a third device relative to the patient support apparatus, and to use the third position and not the first position to determine whether or not to associate the third device with the patient support apparatus.

In some aspects, the controller is further adapted to associate the third device with the patient support apparatus if the third device is positioned inside of the first volume of space, regardless of the first position of the first device.

According to another aspect of the present disclosure, a patient support apparatus is provided that includes a support surface; a first transceiver coupled to a first location on the patient support apparatus; a second transceiver coupled to a second location on the patient support apparatus; a third transceiver coupled to a third location on the patient support apparatus; and a controller. The controller is adapted to use radio frequency (RF) communication between the first, second, and third transceivers and a first and second device in order to determine a first position of the first device relative to the patient support apparatus and a second position of the second device relative to the patient support apparatus. The controller is further adapted to receive a first ID from the first device and a second ID from the second device and to use the first and second IDs to determine whether or not the first device is adapted to control the second device.

In some aspects, the first device is a temperature management device adapted to deliver temperature controlled fluid to the patient, and the second device is a thermal pad adapted to wrap around a portion of the patient and receive the temperature controlled fluid from the temperature management device.

The controller, in some aspects, is further adapted to use the first ID and the second ID to determine a first association condition and a second association condition, respectively, to use the first position and the first association condition to determine whether or not to associate the first device with the patient support apparatus, and to use the second association condition and the second position to determine whether or not to associate the second device with the patient support apparatus.

In some aspects, the first association condition defines a first volume of space in which the first device must be positioned in order for the controller to associate the first device with the patient support apparatus, and the second association condition defines a second volume of space in which the second device must be position in order for the controller to associate the second device with the patient support apparatus.

The first and second association conditions, in some aspects, are stored in a memory onboard the patient support apparatus and the controller is adapted to select the first and second association conditions based on the first and second IDs.

The first and second association conditions are received from the first and second devices, respectively, in some aspects.

The controller, in some aspects, is further adapted to use RF communication between the first, second, and third transceivers and a third and fourth device to determine a third position of the third device relative to the patient support apparatus and a fourth position of the fourth device relative to the patient support apparatus.

The controller may further be adapted to receive a third ID from the third device and a fourth ID from the fourth device and to use the first ID, second ID, third ID and fourth ID to determine first, second, third, and fourth association conditions, respectively.

In some aspects, the controller is further adapted to use the first, second, third, and fourth association conditions to determine whether or not to associate the first device with the patient support apparatus.

In some aspects, the first association condition defines a first volume of space inside which the first device must be positioned in order for the controller to associate the first device with the patient support apparatus.

Before the various aspects of the disclosure are explained in detail, it is to be understood that the claims are not to be limited to the details of operation or to the details of construction and the arrangement of the components set forth in the following description or illustrated in the drawings. The aspects described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus and a patient temperature management device according to a first aspect of the present disclosure;

FIG. 2 is a plan view of an illustrative caregiver control panel of the patient support apparatus of FIG. 1;

FIG. 3 is a plan view of an illustrative patient control panel of the patient support apparatus of FIG. 1;

FIG. 4 is a perspective view of the patient temperature management device of FIG. 1;

FIG. 5 is a block diagram of the patient temperature management device;

FIG. 6 is a diagram of the patient support apparatus, the patient temperature management device, a display device, a fixed locator unit, and several components of a healthcare facility local area network;

FIG. 7 is a block diagram of the patient support apparatus, the patient temperature management device, the fixed locator unit, the display, and a thermal wrap;

FIG. 8 is a table of association information used by the patient support apparatus according to one aspect of the present disclosure; and

FIG. 9 is front view of a display device illustrating several examples of the type of data that may be sent from the patient support apparatus to the display device for displaying thereon.

### DETAILED DESCRIPTION OF THE EMBODIMENTS

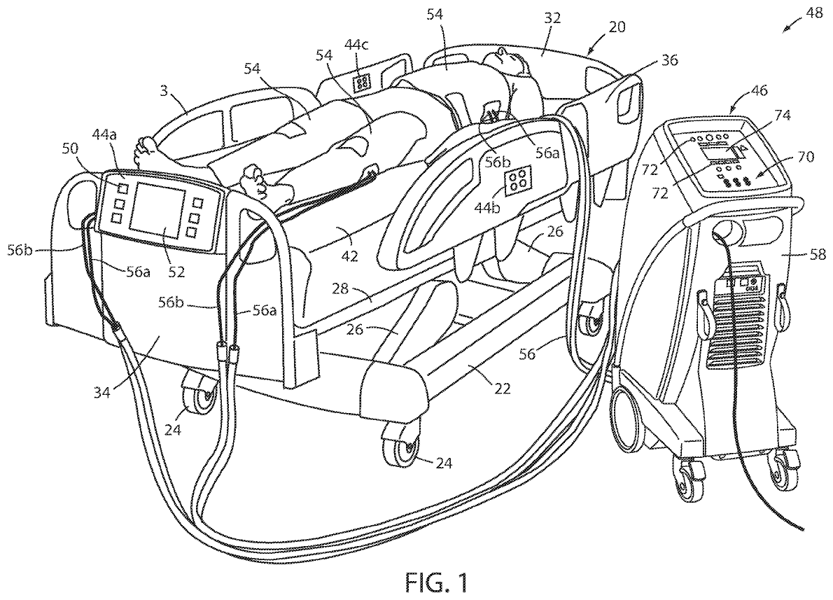

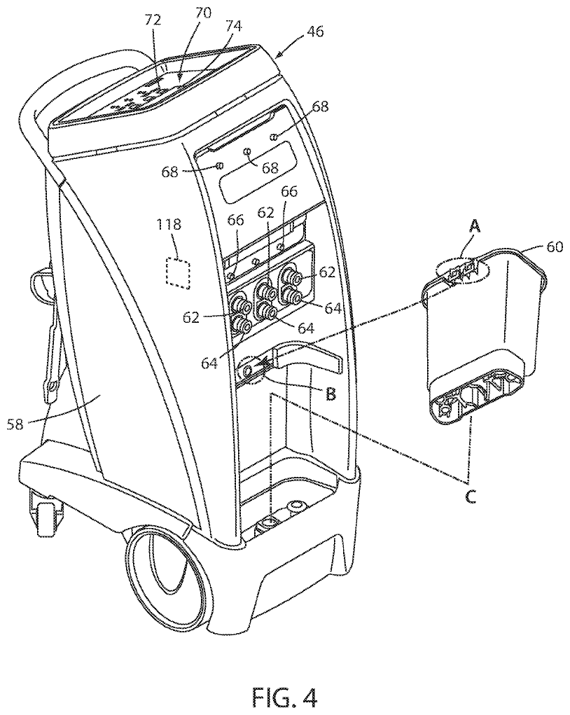

An illustrative patient support apparatus 20 and a thermal control system 48 according to several aspects of the present disclosure are shown in FIG. 1. The thermal control system 48 is used to control the temperature of a patient while he or she is positioned on patient support apparatus 20, and further details of this system 48 are provided below in the discussion of FIGS. 4-7. Although the particular form of patient support apparatus 20 illustrated in FIG. 1 is a bed adapted for use in a hospital or other medical setting, it will be understood that patient support apparatus 20 could, in different embodiments, be a cot, a stretcher, a recliner, an operating table, or any other structure capable of supporting a patient in a healthcare environment.

In general, patient support apparatus 20 includes a base 22 having a plurality of wheels 24, a pair of lifts 26 supported on the base 22, a litter frame 28 supported on the lifts 26, and a support deck 30 supported on the litter frame 28. Patient support apparatus 20 further includes a headboard 32, a footboard 34 and a plurality of siderails 36. Siderails 36 are all shown in a raised position in FIG. 1 but are each individually movable to a lower position in which ingress into, and egress out of, patient support apparatus 20 is not obstructed by the lowered siderails 36.

Lifts 26 are adapted to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted, to place the litter frame 28 in a flat or horizontal orientation, a Trendelenburg orientation, or a reverse Trendelenburg orientation. That is, litter frame 28 includes a head end 38 and a foot end 40, each of whose height can be independently adjusted by the nearest lift 26. Patient support apparatus 20 is designed so that when an occupant lies thereon, his or her head will be positioned adjacent head end 38 and his or her feet will be positioned adjacent foot end 40.

Litter frame 28 provides a structure for supporting support deck 30, the headboard 32, footboard 34, and siderails 36. Support deck 30 provides a support surface for a mattress 42, or other soft cushion, so that a person may lie and/or sit thereon. In some embodiments, the mattress 42 includes one or more inflatable bladders that are controllable via a blower, or other source of pressurized air. In at least one embodiment, the inflation of the bladders of the mattress 42 is controllable via electronics built into patient support apparatus 20. In one such embodiments, mattress 42 may take on any of the functions and/or structures of any of the mattresses disclosed in commonly assigned U.S. Pat. No. 9,468,307 issued Oct. 18, 2016, to inventors Patrick Lafleche et al., the complete disclosure of which is incorporated herein by reference. Still other types of mattresses may be used.

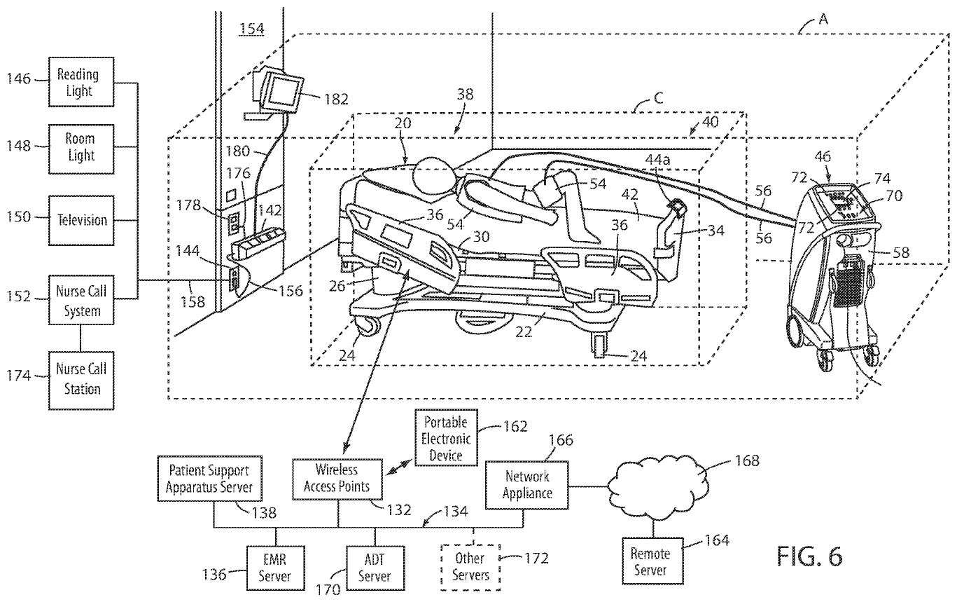

Support deck 30 is made of a plurality of sections, some of which are pivotable about generally horizontal pivot axes. In the embodiment shown in FIG. 1, support deck 30 includes at least a head section, a thigh section, and a foot section, all of which are positioned underneath mattress 42 and which generally form flat surfaces for supporting mattress 42. The head section, which is also sometimes referred to as a Fowler section, is pivotable about a generally horizontal pivot axis between a generally horizontal orientation (shown in FIG. 1) and a plurality of raised positions (one of which is shown in FIG. 6). The thigh section and foot section may also be pivotable about generally horizontal pivot axes.

In some embodiments, patient support apparatus 20 may be modified from what is shown to include one or more components adapted to allow the user to extend the width of patient support deck 30, thereby allowing patient support apparatus 20 to accommodate patients of varying sizes. When so modified, the width of deck 30 may be adjusted sideways in any increments, for example between a first or minimum width, a second or intermediate width, and a third or expanded/maximum width.

It will be understood by those skilled in the art that patient support apparatus 20 can be designed with other types of mechanical constructions that are different from what is shown in the attached drawings, such as, but not limited to, the construction described in commonly assigned, U.S. Pat. No. 10,130,536 to Roussy et al., entitled PATIENT SUPPORT USABLE WITH BARIATRIC PATIENTS, the complete disclosure of which is incorporated herein by reference. In another embodiment, the mechanical construction of patient support apparatus 20 may include the same, or nearly the same, structures as the Model 3002 S3 bed manufactured and sold by Stryker Corporation of Kalamazoo, Michigan. This construction is described in greater detail in the Stryker Maintenance Manual for the MedSurg Bed, Model 3002 S3, published in 2010 by Stryker Corporation of Kalamazoo, Michigan, the complete disclosure of which is incorporated herein by reference. In still another embodiment, the mechanical construction of patient support apparatus 20 may include the same, or nearly the same, structure as the Model 3009 Procuity MedSurg bed manufactured and sold by Stryker Corporation of Kalamazoo, Michigan. This construction is described in greater detail in the Stryker Maintenance Manual for the 3009 Procuity MedSurg bed (publication 3009-009-002, Rev. A.0), published in 2020 by Stryker Corporation of Kalamazoo, Michigan.

It will be understood by those skilled in the art that patient support apparatus 20 can be designed with still other types of mechanical constructions, such as, but not limited to, those described in commonly assigned, U.S. Pat. No. 7,690,059 issued Apr. 6, 2010, to Lemire et al., and entitled HOSPITAL BED; and/or commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The overall mechanical construction of patient support apparatus 20 may also take on still other forms different from what is disclosed in the aforementioned references provided the patient support apparatus includes one or more of the functions, features, and/or structures discussed in greater detail below.

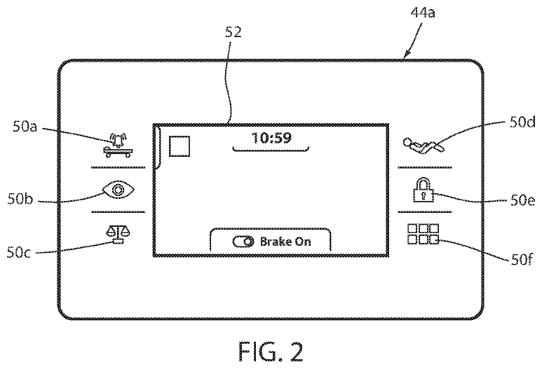

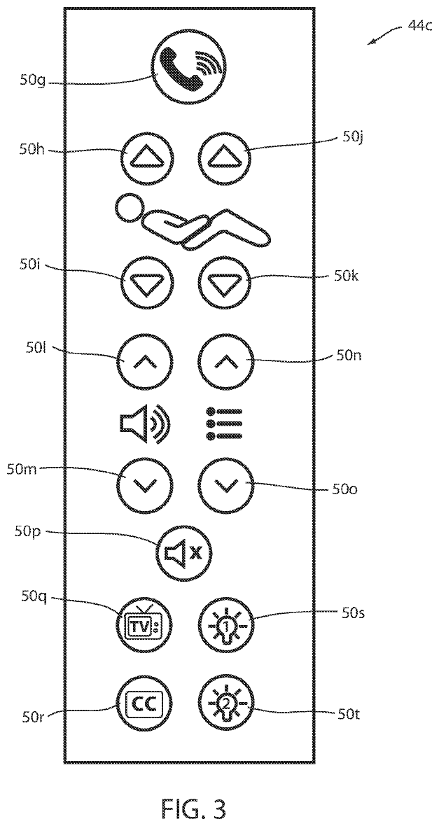

Patient support apparatus 20 further includes a plurality of control panels 44 that enable a user of patient support apparatus 20, such as a patient and/or an associated caregiver, to control one or more aspects of patient support apparatus 20. In the embodiment shown in FIG. 1, patient support apparatus 20 includes a footboard control panel 44a, a pair of outer siderail control panels 44b (only one of which is visible), and a pair of inner siderail control panels 44c (only one of which is visible). Footboard control panel 44a and outer siderail control panels 44b are intended to be used by caregivers, or other authorized personnel, while inner siderail control panels 44c are intended to be used by the patient associated with patient support apparatus 20. Each of the control panels 44 includes a plurality of controls 50 (see, e.g. FIGS. 2-3), although each control panel 44 does not necessarily include the same controls and/or functionality.

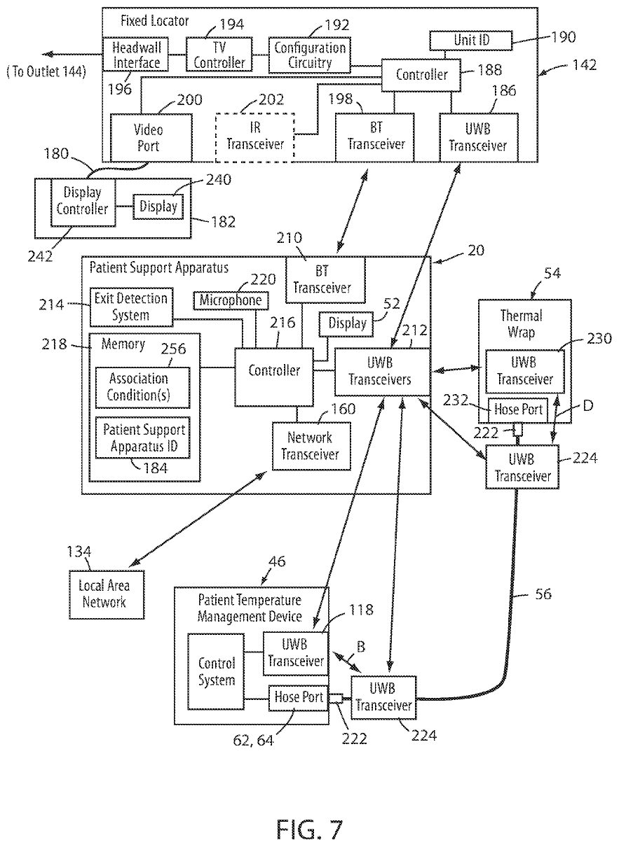

Among other functions, controls 50 of control panel 44a allow a user to control one or more of the following: change a height of support deck 30, raise or lower the Fowler section, activate and deactivate a brake for wheels 24, arm and disarm an exit detection system 214 (FIG. 7), change various settings on patient support apparatus 20, view the current location of the patient support apparatus 20 as determined by the location detection system discussed herein, view data from a patient temperature management device 46 (FIGS. 1 & 4-7) used with the patient, view what medical devices—if any—the patient support apparatus 20 has associated itself with, control what information from the patient temperature management device 46 is displayed and/or sent to an Electronic Medical Records (EMR) server, and perform other actions. One or both of the inner siderail control panels 44c also include at least one control that enables a patient to call a remotely located nurse (or other caregiver). In addition to the nurse call control, one or both of the inner siderail control panels 44c also include one or more controls for controlling one or more features of one or more room devices positioned within the same room as the patient support apparatus 20. As will be described in more detail below, such room devices include, but are not necessarily limited to, a television, a reading light, and a room light. With respect to the television, the features that may be controllable by one or more controls 50 on control panel 44c include, but are not limited to, the volume, the channel, the closed-captioning, and/or the power state of the television. With respect to the room and/or night lights, the features that may be controlled by one or more controls 50 on control panel 44c include the on/off state and/or the brightness level of these lights.

Control panel 44a includes a display 52 (FIG. 2) configured to display a plurality of different screens thereon. Surrounding display 52 are a plurality of navigation controls 50a-f that, when activated, cause the display 52 to display different screens on display 52. More specifically, when a user presses navigation control 50a, control panel 44a displays an exit detection control screen on display 52 that includes one or more icons that, when touched, control the onboard exit detection system 214 (FIG. 7). The exit detection system 214 is as adapted to issue an alert when a patient exits from patient support apparatus 20. Exit detection system 214 may include any of the same features and functions as, and/or may be constructed in any of the same manners as, the exit detection system disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, the complete disclosure of which is incorporated herein by reference. Other types of exit detection systems may be included within patient support apparatus 20.

When a user presses navigation control 50b (FIG. 2), control panel 44 displays a monitoring control screen that includes a plurality of control icons that, when touched, control an onboard monitoring system built into patient support apparatus 20. The onboard monitoring system alerts the caregiver through a unified indicator, such as a light or a plurality of lights controlled in a unified manner, when any one or more of a plurality of settings on patient support apparatus 20 are in an undesired state, and uses that same unified indicator to indicate when all of the plurality of settings are in their respective desired states. Further details of one type of monitoring system that may be built into patient support apparatus 20 are disclosed in commonly assigned U.S. patent application Ser. No. 62/864,638 filed Jun. 21, 2019, by inventors Kurosh Nahavandi et al. and entitled PATIENT SUPPORT APPARATUS WITH CAREGIVER REMINDERS, as well as commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosures of both of which are incorporated herein by reference. Other types of monitoring systems may be included within patient support apparatus 20.

When a user presses navigation control 50c, control panel 44a displays a scale control screen that includes a plurality of control icons that, when touched, control the scale system of patient support apparatus 20. Such a scale system may include any of the same features and functions as, and/or may be constructed in any of the same manners as, the scale systems disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, and U.S. patent application Ser. No. 62/885,954 filed Aug. 13, 2019, by inventors Kurosh Nahavandi et al. and entitled PATIENT SUPPORT APPARATUS WITH EQUIPMENT WEIGHT LOG, the complete disclosures of both of which are incorporated herein by reference. The scale system may utilize the same force sensors that are utilized by the exit detection system 214, or it may utilize one or more different sensors. Other scale systems besides those mentioned above in the '254 and '954 applications may alternatively be included within patient support apparatus 20.

When a user presses navigation control 50d, control panel 44 displays a motion control screen that includes a plurality of control icons that, when touched, control the movement of various components of patient support apparatus 20, such as, but not limited to, the height of litter frame 28 and the pivoting of the Fowler section. In some embodiments, the motion control screen displayed on display 52 in response to pressing control 50d may be the same as, or similar to, the position control screen 216 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of motion control screens may be included on patient support apparatus 20.

When a user presses navigation control 50e, control panel 44a displays a motion lock control screen that includes a plurality of control icons that, when touched, control one or more motion lockout functions of patient support apparatus 20. Such motion lockout functions typically include the ability for a caregiver to use control panel 44a to lock out one or more of the motion controls 50 of the patient control panels 44c such that the patient is not able to use those controls 50 on control panels 44c to control the movement of one or more components of patient support apparatus 20. The motion lockout screen may include any of the features and functions as, and/or may be constructed in any of the same manners as, the motion lockout features, functions, and constructions disclosed in commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosure of which is incorporated herein by reference. Other types of motion lockouts may be included within patient support apparatus 20.

When a user presses on navigation control 50f, control panel 44a displays a menu screen that includes a plurality of menu icons that, when touched, bring up one or more additional screens for controlling and/or viewing one or more other aspects of patient support apparatus 20. Such other aspects include, but are not limited to, displaying information about one or more medical devices that are currently associated with patient support apparatus 20 (e.g. a patient temperature management device, one or more vital sign sensors, etc.), diagnostic and/or service information for patient support apparatus 20, mattress control and/or status information, configuration settings, location information, and other settings and/or information. One example of a suitable menu screen is the menu screen 100 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of menus and/or settings may be included within patient support apparatus 20.

In at least one embodiment, utilization of navigation control 50f allows a user to navigate to a screen that enables a user to see which medical devices, if any, are currently associated with patient support apparatus 20. In some embodiments, as will be discussed in greater detail herein, patient support apparatus 20 is configured to automatically associate one or more devices with itself when those devices meet certain association conditions, such as being located within a predefined volume of space that encompasses and surrounds patient support apparatus 20. As will be discussed in greater detail below, patient support apparatus 20 includes an onboard locating system that is adapted to automatically determine the relative position of one or more devices with respect to patient support apparatus 20 and, in some instances, automatically associate those devices with patient support apparatus 20 (and/or the patient assigned to patient support apparatus 20) depending upon the proximity of the medical device to patient support apparatus 20 and/or other criteria Further details of this locating system and association process are provided below.

For all of the navigation controls 50a-f (FIG. 2), screens other than the ones specifically mentioned above may be displayed on display 52 in other embodiments of patient support apparatus 20 in response to a user pressing these controls. Thus, it will be understood that the specific screens mentioned above are merely representative of the types of screens that are displayable on display 52 in response to a user pressing on one or more of navigation controls 50a-f. It will also be understood that, although navigation controls 50a-f have all been illustrated in the accompanying drawings as dedicated controls that are positioned adjacent display 52, any one or more of these controls 50a-f could alternatively be touchscreen controls that are displayed at one or more locations on display 52. Still further, although controls 50a-f have been shown herein as buttons, it will be understood that any of controls 50a-f could also, or alternatively, be switches, dials, or other types of non-button controls. Additionally, patient support apparatus 20 may be modified to include additional, fewer, and/or different navigation controls from the navigation controls 50a-f shown in FIG. 2.

FIG. 3 illustrates one example of a patient control panel 44c that may be incorporated into patient support apparatus 20 and positioned at a location on patient support apparatus 20 that is convenient for a patient to access while supported on support deck 30, such as on an interior side of one of the siderails 36. Control panel 44c includes a plurality of controls 50g-t that are intended to be operated by a patient. A nurse call control 50g, when pressed by the patient, sends a signal to a nurse call system requesting that a remotely positioned nurse talk to the patient. A Fowler-up control 50h, when pressed by the patient, causes a motorized actuator onboard patient support apparatus 20 to raise the Fowler section upwardly. A Fowler-down control 50i, when pressed by the patient, causes the motorized actuator to lower the Fowler section downwardly. A gatch-up control 50j, when pressed by the patient, causes another motorized actuator to raise a knee section of support deck 30, while a gatch-down control 50k causes the motorized actuator to lower the knee section of support deck 30.

A volume-up control 50l, when pressed by the patient, causes patient support apparatus 20 to send a signal to an in-room television instructing it to increase its volume, while a volume down control 50m, when pressed, causes patient support apparatus 20 to send a signal to the television instructing it to decrease its volume. A channel-up control 50n, when pressed by the patient, causes patient support apparatus 20 to send a signal to the television instructing it to increase the channel number, while a channel-down control 50o, when pressed, causes patient support apparatus 20 to send a signal to the television instructing it to decrease the channel number.

A mute control 50p, when pressed, causes patient support apparatus 20 to send a signal to the television instructing it to either mute itself or unmute itself, depending upon whether the television is currently muted or unmuted. In other words, mute control 50p is a toggle control that alternatingly sends mute and unmute commands to the television when it is pressed.

Power control 50q is a toggle control that, when pressed, sends a signal to the television to either turn on or turn off, depending upon the television's current power status. Closed-captioning control 50r is another toggle control that, when pressed, sends a signal to the television to either turn on its closed-captioning feature or to turn off its closed captioning feature, depending upon whether the closed-captioning feature is currently on or off.

Control 50s is a toggle control that, when pressed, sends a signal to a first light to either turn on or turn off, depending upon the current state of that first light. Control 50t is another toggle control that, when pressed, sends a signal to a second light to either turn on or turn off, depending upon the current state of that second light. In some embodiments, the first light is a reading light and the second light is a room light, both of which are positioned off-board the patient support apparatus 20.

It will be understood that not only the number of controls 50 on control panel 44c, but also the functions of the controls 50 on control panel 44c, the layout of the controls 50 on control panel 44c, and/or other aspects of control panel 44c may be modified from what is shown in FIG. 3. In some embodiments, control panel 44c is implemented on a pendant controller that includes a cable that is plugged into a port on patient support apparatus 20. In other embodiments, one or more of the controls 50 of control panel 44c may be omitted, augmented, and/or split amongst other controls panels and/or locations. Still other manners of implementing control panel 44c are also possible.

Thermal control system 48 (FIG. 1) is adapted to control the temperature of a patient, which may involve raising, lowering, and/or maintaining the patient's temperature. Thermal control system 48 includes a patient temperature management device 46 coupled to one or more thermal therapy devices 54. The thermal therapy devices 54 are illustrated in FIG. 1 to be thermal wraps, but it will be understood that thermal therapy devices 54 may take on other forms, such as, but not limited to, blankets, vests, patches, caps, catheters, or other structures that receive temperature-controlled fluid. For purposes of the following written description, thermal therapy devices 54 will be referred to as thermal wraps 54, but it will be understood by those skilled in the art that this terminology is used merely for convenience and that the phrase “thermal wrap” is intended to cover all of the different variations of thermal therapy devices 54 mentioned above (e.g. blankets, vests, patches, caps, catheters, etc.) and variations thereof.

Patient temperature management device 46 is coupled to thermal wraps 54 via a plurality of hoses 56. Patient temperature management device 46 delivers temperature-controlled fluid (such as, but not limited to, water or a water mixture) to the thermal wraps 54 via the fluid supply hoses 56. After the temperature-controlled fluid has passed through thermal wraps 54, patient temperature management device 46 receives the temperature-controlled fluid back from thermal wraps 54 via the return hoses 56b.

In the embodiment of thermal control system 48 shown in FIG. 1, three thermal wraps 54 are used in the treatment of the patient. A first thermal wrap 54 is wrapped around a patient's torso, while second and third thermal wraps 54 are wrapped, respectively, around the patient's right and left legs. Other configurations can be used and different numbers of thermal wraps 54 may be used with patient temperature management device 46, depending upon the number of inlet and outlet ports that are included with patient temperature management device 46. By controlling the temperature of the fluid delivered to thermal wraps 54 via supply hoses 56a, the temperature of the patient can be controlled via the close contact of the wraps 54 with the patient and the resultant heat transfer therebetween.

As shown more clearly in FIG. 4, patient temperature management device 46 includes a main body 58 to which a removable reservoir 60 may be coupled and uncoupled. Removable reservoir 60 is configured to hold the fluid that is to be circulated through patient temperature management device 46 and the one or more thermal wraps 54. By being removable from patient temperature management device 46, reservoir 60 can be easily carried to a sink or faucet for filling and/or dumping of the water or other fluid. This allows users of thermal control system 48 to more easily fill patient temperature management device 46 prior to its use, as well as to drain patient temperature management device 46 after use.

As can also be seen in FIG. 4, patient temperature management device 46 includes a plurality of outlet ports 62 (three in the particular example of FIG. 4), a plurality of inlets ports 64 (three in this particular example). Outlet ports 62 are adapted to fluidly couple to supply hoses 56a and inlet ports 64 are adapted to fluidly couple to return hoses 56b. Patient temperature management device 46 also includes a plurality of patient temperature probe ports 66, a plurality of auxiliary ports 68, and a control panel 70 having a plurality of dedicated controls 72 and a display 74 (see also FIGS. 1 & 5). The patient temperature probe ports 66, auxiliary ports 68, and control panel 70 are described in more detail below.

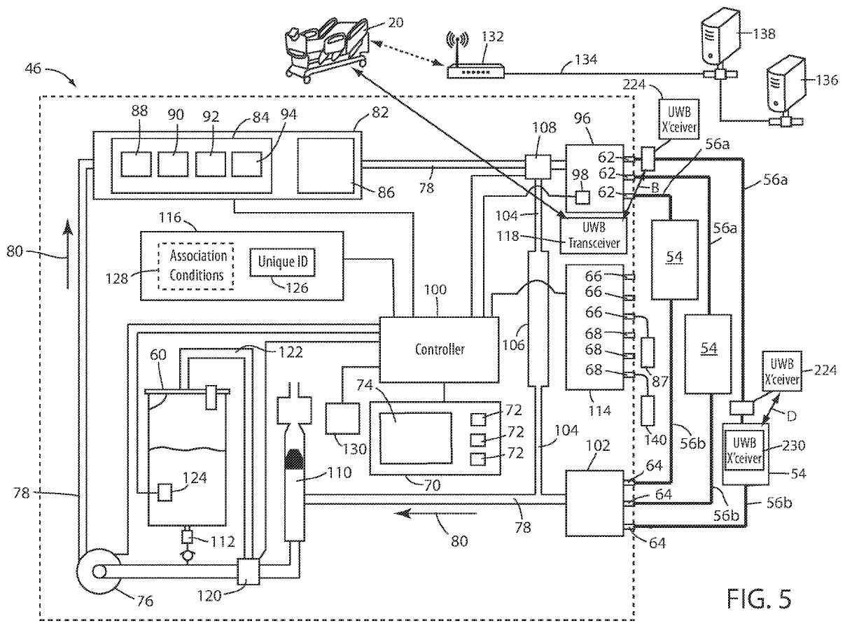

As shown in FIG. 5, patient temperature management device 46 includes a pump 76 for circulating fluid through a circulation channel 78. Pump 76, when activated, circulates the fluid through circulation channel 78 in the direction of arrows 80 (clockwise in FIG. 5). Starting at pump 76 the circulating fluid first passes through a heat exchanger 82 that adjusts, as necessary, the temperature of the circulating fluid. Heat exchanger 82 may take on a variety of different forms. In some embodiments, heat exchanger 82 is a thermoelectric heater and cooler. In the embodiment shown in FIG. 5, heat exchanger 82 includes a chiller 84 and a heater 86. Further, in the embodiment shown in FIG. 5, chiller 84 is a conventional vapor-compression refrigeration unit having a compressor 88, a condenser 90, an evaporator 92, an expansion valve (not shown), and a fan 94 for removing heat from the compressor 88. Heater 86 is a conventional electrical resistance-based heater. Other types of chillers and/or heaters may be used.

After passing through heat exchanger 82, the circulating fluid is delivered to an outlet manifold 96 having an outlet temperature sensor 98 and a plurality of outlet ports 62. Temperature sensor 98 is adapted to detect a temperature of the fluid inside of outlet manifold 96 and report it to a controller 100. Outlet ports 62 are adapted to releasably couple to supply hoses 56a. Supply hoses 56a are adapted to releasably couple, in turn, to thermal wraps 54 and deliver temperature-controlled fluid to the thermal wraps 54. The temperature-controlled fluid, after passing through the thermal wraps 54, is returned to patient temperature management device 46 via return hoses 56b. Return hoses 56b are adapted to be releasably coupled to a plurality of inlets ports 64. The releasable coupling between hoses 56, ports 62 and 64 of patient temperature management device 46, and thermal wraps 54 may take place via any suitable means, such as a conventional Colder-type connector, or another type of connector. Inlets ports 64 are fluidly coupled to an inlet manifold 102 inside of patient temperature management device 46.

Patient temperature management device 46 also includes a bypass line 104 fluidly coupled to outlet manifold 96 and inlet manifold 102 (FIG. 5). Bypass line 104 allows fluid to circulate through circulation channel 78 even in the absence of any thermal wraps 54 or hoses 56a being coupled to any of outlet ports 62. In the illustrated embodiment, bypass line 104 includes a filter 106 that is adapted to filter the circulating fluid. If included, filter 106 may be a particle filter adapted to filter out particles within the circulating fluid that exceed a size threshold, or filter 106 may be a biological filter adapted to purify or sanitize the circulating fluid, or it may be a combination of both. In some embodiments, filter 106 is constructed and/or positioned within patient temperature management device 46 in any of the manners disclosed in commonly assigned U.S. patent application Ser. No. 62/404,676 filed Oct. 11, 2016, by inventors Marko Kostic et al. and entitled THERMAL CONTROL SYSTEM, the complete disclosure of which is incorporated herein by reference.

The flow of fluid through bypass line 104 is controllable by way of a bypass valve 108 positioned at the intersection of bypass line 104 and outlet manifold 96 (FIG. 5). When open, bypass valve 108 allows fluid to flow through circulation channel 78 to outlet manifold 96, and from outlet manifold 96 to the connected thermal wraps 54. When closed, bypass valve 108 stops fluid from flowing to outlet manifold 96 (and thermal wraps 54) and instead diverts the fluid flow along bypass line 104. In some embodiments, bypass valve 108 may be controllable by controller 100 such that selective portions of the fluid are directed to outlet manifold 96 and along bypass line 104. In some embodiments, bypass valve 108 is controlled in any of the manners discussed in commonly assigned U.S. patent application Ser. No. 62/610,319, filed Dec. 26, 2017, by inventors Gregory Taylor et al. and entitled THERMAL SYSTEM WITH OVERSHOOT REDUCTION, the complete disclosure of which is incorporated herein by reference. In other embodiments, bypass valve 108 may be a pressure operated valve that allows fluid to flow along bypass line 104 if the fluid pressure in circulation channel 78 exceeds the cracking pressure of the bypass valve 108. Still further, in some embodiments, bypass valve 108 may be omitted and fluid may be allowed to flow through both bypass line 104 and into outlet manifold 96.

The incoming fluid flowing into inlet manifold 102 from inlets ports 64 and/or bypass line 104 travels back toward pump 76 and into an air remover 110. Air remover 110 includes any structure in which the flow of fluid slows down sufficiently to allow air bubbles contained within the circulating fluid to float upwardly and escape to the ambient surroundings. In some embodiments, air remover 110 is constructed in accordance with any of the configurations disclosed in commonly assigned U.S. patent application Ser. No. 15/646,847 filed Jul. 11, 2017, by inventor Gregory S. Taylor and entitled THERMAL CONTROL SYSTEM, the complete disclosure of which is hereby incorporated herein by reference. After passing through air remover 110, the circulating fluid flows past a valve 112 positioned beneath fluid reservoir 60. Fluid reservoir 60 supplies fluid to patient temperature management device 46 and circulation channel 78 via valve 112, which may be a conventional check valve, or other type of valve, that automatically opens when reservoir 60 is coupled to patient temperature management device 46 and that automatically closes when reservoir 60 is decoupled from patient temperature management device 46 (see FIG. 4). After passing by valve 112, the circulating fluid travels to pump 76 and the fluid circuit is repeated.

Controller 100 of patient temperature management device 46 is contained within main body 58 of patient temperature management device 46 and is in electrical communication with pump 76, heat exchanger 82, outlet temperature sensor 98, bypass valve 108, a sensor module 114, control panel 70, a memory 116, a network transceiver 130, and one or more ultra-wideband transceivers 118. Controller 100 includes any and all electrical circuitry and components necessary to carry out the functions and algorithms described herein, as would be known to one of ordinary skill in the art. Generally speaking, controller 100 may include one or more microcontrollers, microprocessors, and/or other programmable electronics that are programmed to carry out the functions described herein. It will be understood that controller 100 may also include other electronic components that are programmed to carry out the functions described herein, or that support the microcontrollers, microprocessors, and/or other electronics. The other electronic components include, but are not limited to, one or more field programmable gate arrays, systems on a chip, volatile or nonvolatile memory, discrete circuitry, integrated circuits, application specific integrated circuits (ASICs) and/or other hardware, software, or firmware, as would be known to one of ordinary skill in the art. Such components can be physically configured in any suitable manner, such as by mounting them to one or more circuit boards, or arranging them in other manners, whether combined into a single unit or distributed across multiple units. Such components may be physically distributed in different positions in patient temperature management device 46, or they may reside in a common location within patient temperature management device 46. When physically distributed, the components may communicate using any suitable serial or parallel communication protocol, such as, but not limited to, CAN, LIN, Firewire, I-squared-C, RS-232, RS-465, universal serial bus (USB), etc.

Control panel 70 allows a user to operate patient temperature management device 46. Control panel 70 communicates with controller 100 and includes a display 74 and a plurality of dedicated controls 72a, 72b, 72c, etc. Display 74 may be implemented as a touch screen, or, in other embodiments, as a non-touch-sensitive display. Dedicated controls 72 may be implemented as buttons, switches, dials, or other dedicated structures. In any of the embodiments, one or more of the functions carried out by a dedicated control 72 may be replaced or supplemented with a touch screen control that is activated when touched by a user. Alternatively, in any of the embodiments, one or more of the controls that are carried out via a touch screen can be replaced or supplemented with a dedicated control 72 that carries out the same function when activated by a user.

Through either dedicated controls 72 and/or a touch screen display (e.g. display 74), control panel 70 enables a user to turn patient temperature management device 46 on and off, select a mode of operation, select a target temperature for the fluid delivered to thermal wraps 54, select a patient target temperature, customize a variety of treatment, display, alarm, and other functions, view the association status of patient temperature management device 46 with patient support apparatus 20 and/or with hoses 56 and/or thermal wraps 54, control what information from patient temperature management device 46 is sent to patient support apparatus 20, and control still other aspects of patient temperature management device 46, as is discussed in greater detail below. In some embodiments, control panel 70 may include a pause/event control, a medication control, and/or an automatic temperature adjustment control that operate in accordance with the pause event control 66b, medication control 66c, and automatic temperature adjustment control 66d disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference. Such controls may be activated as touch screen controls or dedicated controls 72.

In those embodiments where control panel 70 allows a user to select from different modes for controlling the patient's temperature, the different modes include, but are not limited to, a manual mode and an automatic mode, both of which may be used for cooling and heating the patient. In the manual mode, a user selects a target temperature for the fluid that circulates within patient temperature management device 46 and that is delivered to thermal wraps 54. Patient temperature management device 46 then makes adjustments to heat exchanger 82 in order to ensure that the temperature of the fluid exiting supply hoses 56a is at the user-selected temperature.

When the user selects the automatic mode, the user selects a target patient temperature, rather than a target fluid temperature. After selecting the target patient temperature, controller 100 makes automatic adjustments to the temperature of the fluid in order to bring the patient's temperature to the desired patient target temperature. In this mode, the temperature of the circulating fluid may vary as necessary in order to bring about the target patient temperature.

In order to carry out the automatic mode, patient temperature management device 46 utilizes a sensor module 114 that includes one or more patient temperature sensor ports 66 (FIGS. 4 & 5) that are adapted to receive one or more conventional patient temperature sensors or probes 87. The patient temperature sensors 87 may be any suitable patient temperature sensor that is able to sense the temperature of the patient at the location of the sensor. In one embodiment, the patient temperature sensors are conventional Y.S.I. 400 probes marketed by YSI Incorporated of Yellow Springs, Ohio, or probes that are YSI 400 compliant or otherwise marketed as 400 series probes. In other embodiments, different types of sensors may be used with patient temperature management device 46. Regardless of the specific type of patient temperature sensor used in thermal control system 48, each temperature sensor 87 is connected to a patient temperature sensor port 66 positioned on patient temperature management device 46. Patient temperature sensor ports 66 are in electrical communication with controller 100 and provide current temperature readings of the patient's temperature.

Controller 100, in some embodiments, controls the temperature of the circulating fluid using closed-loop feedback from temperature sensor 98 (and, when operating in the automatic mode, also from patient temperature sensor(s) 87). That is, controller 100 determines (or receives) a target temperature of the fluid, compares it to the measured temperature from sensor 98, and issues a command to heat exchanger 82 that seeks to decrease the difference between the desired fluid temperature and the measured fluid temperature. In some embodiments, the difference between the fluid target temperature and the measured fluid temperature is used as an error value that is input into a conventional Proportional, Integral, Derivative (PID) control loop. That is, controller 100 multiplies the fluid temperature error by a proportional constant, determines the derivative of the fluid temperature error over time and multiplies it by a derivative constant, and determines the integral of the fluid temperature error over time and multiplies it by an integral constant. The results of each product are summed together and converted to a heating/cooling command that is fed to heat exchanger 82 and tells heat exchanger 82 whether to heat and/or cool the circulating fluid and how much heating/cooling power to use.

When patient temperature management device 46 is operating in the automatic mode, controller 100 may use a second closed-loop control loop that determines the difference between a patient target temperature and a measured patient temperature. The patient target temperature is input by a user of patient temperature management device 46 using control panel 70. The measured patient temperature comes from a patient temperature sensor 87 coupled to one of patient temperature sensor ports 66 (FIG. 5). Controller 100 determines the difference between the patient target temperature and the measured patient temperature and, in some embodiments, uses the resulting patient temperature error value as an input into a conventional PID control loop. As part of the PID loop, controller 100 multiplies the patient temperature error by a proportional constant, multiplies a derivative of the patient temperature error over time by a derivative constant, and multiplies an integral of the patient temperature error over time by an integral constant. The three products are summed together and converted to a target fluid temperature value. The target fluid temperature value is then fed to the first control loop discussed above, which uses it to compute a fluid temperature error.

It will be understood by those skilled in the art that other types of control loops may be used. For example, controller 100 may utilize one or more PI loops, PD loops, and/or other types of control equations. In some embodiments, the coefficients used with the control loops may be varied by controller 100 depending upon the patient's temperature reaction to the thermal therapy, among other factors. One example of such dynamic control loop coefficients is disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference.

Regardless of the specific control loop utilized, controller 100 implements the loop(s) multiple times a second in at least one embodiment, although it will be understood that this rate may be varied widely. After controller 100 has output a heat/cool command to heat exchanger 82, controller 100 takes another patient temperature reading (from sensor 87) and/or another fluid temperature reading (from sensor 98) and re-performs the loop(s). The specific loop(s) used, as noted previously, depends upon whether patient temperature management device 46 is operating in the manual mode or automatic mode.

It will also be understood by those skilled in the art that the output of any control loop used by patient temperature management device 46 may be limited such that the temperature of the fluid delivered to thermal wraps 54 never strays outside of a predefined maximum and a predefined minimum. Examples of such a predefined maximum temperature and predefined minimum temperature are disclosed and discussed in greater detail in commonly assigned U.S. patent application Ser. No. 16/222,004 filed Dec. 17, 2018, by inventors Gregory S. Taylor et al. and entitled THERMAL SYSTEM WITH GRAPHICAL USER INTERFACE, the complete disclosure of which is incorporated herein by reference. The predefined minimum temperature is designed as a safety temperature and may be set to about four degrees Celsius, although other temperatures may be selected. The predefined maximum temperature is also implemented as a safety measure and may be set to about forty degrees Celsius, although other values may be selected.

In some embodiments of patient temperature management device 46, such as the embodiment shown in FIG. 5, patient temperature management device 46 also includes a reservoir valve 120 that is adapted to selectively move fluid reservoir 60 into and out of line with circulation channel 78. Reservoir valve 120 is positioned in circulation channel 78 between air remover 110 and valve 112, although it will be understood that reservoir valve 120 may be moved to different locations within circulation channel 78. Reservoir valve 120 is coupled to circulation channel 78 as well as a reservoir channel 122. When reservoir valve 120 is open, fluid from air remover 110 flows along circulation channel 78 to pump 76 without passing through reservoir 60 and without any fluid flowing along reservoir channel 122. When reservoir valve 120 is closed, fluid coming from air remover 110 flows along reservoir channel 122, which feeds the fluid into reservoir 60. Fluid inside of reservoir 60 then flows back into circulation channel 78 via valve 112. Once back in circulation channel 78, the fluid flows to pump 76 and is pumped to the rest of circulation channel 78 and thermal wraps 54 and/or bypass line 104. In some embodiments, reservoir valve 120 is either fully open or fully closed, while in other embodiments, reservoir valve 120 may be partially open or partially closed. In either case, reservoir valve 120 is under the control of controller 100.

In those embodiments of patient temperature management device 46 that include a reservoir valve 120, patient temperature management device 46 may also include a reservoir temperature sensor 124. Reservoir temperature sensor 124 reports its temperature readings to controller 100. When reservoir valve 120 is open, the fluid inside of reservoir 60 stays inside of reservoir 60 (after the initial drainage of the amount of fluid needed to fill circulation channel 78 and thermal wraps 54). This residual fluid is substantially not affected by the temperature changes made to the fluid within circulation channel 78 as long as reservoir valve 120 remains open. This is because the residual fluid that remains inside of reservoir 60 after circulation channel 78 and thermal wraps 54 have been filled does not pass through heat exchanger 82 and remains substantially thermally isolated from the circulating fluid. Two results flow from this: first, heat exchanger 82 does not need to expend energy on changing the temperature of the residual fluid in reservoir 60, and second, the temperature of the circulating fluid in circulation channel 78 will deviate from the temperature of the residual fluid as the circulating fluid circulates through heat exchanger 82.

In some embodiments, controller 100 utilizes a temperature control algorithm to control reservoir valve 120 that, in some embodiments, is the same as the temperature control algorithm 160 disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference. In other embodiments, controller 100 utilizes a different control algorithm. In still other embodiments, patient temperature management device 46 is modified to omit reservoir valve 120, reservoir channel 122, and reservoir temperature sensor 124. Patient temperature management device 46 may also be modified such that reservoir 60 is always in the path of circulation channel 78. Still other modifications are possible.

It will be understood that the particular order of the components along circulation channel 78 of patient temperature management device 46 may be varied from what is shown in FIG. 5. For example, although FIG. 5 depicts pump 76 as being upstream of heat exchanger 82 and air separator 70 as being upstream of pump 76, this order may be changed. Air separator 110, pump 76, heat exchanger 82 and reservoir 60 may be positioned at any suitable location along circulation channel 78. Indeed, in some embodiments, reservoir 60 is moved so as to be in line with and part of circulation channel 78, rather than external to circulation channel 78 as shown in FIG. 5, thereby forcing the circulating fluid to flow through reservoir 60 rather than around reservoir 60. It will also be understood that patient temperature management device 46 does not need to include all of the components shown in FIG. 5, and that many embodiments of patient temperature management device 46 may be implemented in accordance with the present disclosure that omit one or more of these illustrated components. Further details regarding the construction and operation of one embodiment of patient temperature management device 46 that are not described herein may be found in commonly assigned U.S. patent application Ser. No. 14/282,383 filed May 20, 2014, by inventors Christopher Hopper et al. and entitled THERMAL CONTROL SYSTEM, the complete disclosure of which is incorporated herein by reference.

In some embodiments, thermal wraps 54 (FIGS. 1, 5-7) are constructed in accordance with any of the thermal pads disclosed in any of the following commonly assigned U.S. patent applications: Ser. No. 15/675,061 filed Aug. 11, 2017, by inventors James Galer et al. and entitled THERMAL THERAPY DEVICES; Ser. No. 62/778,034 filed Dec. 11, 2018, by inventors Andrew M. Bentz et al. and entitled THERMAL SYSTEM WITH THERMAL PAD FILTERS; and Ser. No. 15/675,066 filed Aug. 11, 2017, by inventor James K. Galer and entitled THERMAL SYSTEM, the complete disclosures of all of which are incorporated herein by reference. Still other types of thermal wraps 54 may be used with thermal control system 48, and patient temperature management device 46 may be modified from its construction described herein in order to accommodate the particular thermal therapy pad(s) it is used with.

Memory 116 (FIG. 5) may be any type of conventional non-volatile memory, such as, but not limited to flash memory, one or more hard drives, one or more EEPROMs, etc. Memory 116 may also be implemented to include more than one of these types of memories in combination. In the embodiment shown in FIG. 5, memory 116 of patient temperature management device 46 includes, among other information, a unique ID 126 and one or more association conditions 128. Unique ID 126 uniquely identifies patient temperature management device 46 and distinguishes it from any other patient temperature management devices 46 that may be present within a given healthcare facility. Association conditions are used by controller 100, in some embodiments, to determine whether a hose 56 should be associated with patient temperature management device 46 or not, as will be discussed in greater detail below. Association conditions 128 are able to be entered into memory 116 locally via control panel 70 and/or are written into memory 116 by controller 100. Additionally, in some embodiments, association conditions 128 may be transferred (wired or wirelessly) to patient temperature management device 46 from another device, such as, but not limited to, a server, a flash drive, a smart phone, etc. Such data transfers may take place via a UWB transceiver 118 and/or via another transceiver, such as, but not limited to, network transceiver 130 and/or a Bluetooth radio. Memory 116 may also include additional information beyond that shown in FIG. 5, such as, but not limited to, one or more algorithms for carrying out its functions, data recorded during the operation of patient temperature management device 46, and/or other data. Memory 116 may also, in some embodiments, omit the association conditions 128, in which case patient support apparatus 20 and/or patient support apparatus server 138 makes the determination of whether a hose 56 is to be associated with the patient temperature management device 46, as will be discussed in more detail below.

Network transceiver 130 is adapted to communicate with one or more off-board devices, such as, but not limited to, a wireless access point of a local area network, a network cable of a local area network, and/or other devices. In the embodiment shown in FIG. 5, transceiver 130 is a Wi-Fi radio communication module configured to wirelessly communicate with one or more wireless access points 132 of a local area network 134. In such embodiments, transceiver 130 may operate in accordance with any of the various IEEE 802.11 standards (e.g. 802.11b, 802.11n, 802.11g, 802.11ac, 802.11ah, etc.). In other embodiments, transceiver 130 may include, either additionally or in lieu of the Wi-Fi radio and communication module, a wired port for connecting a network wire to patient temperature management device 46. In some such embodiments, the wired port accepts a category 5e cable (Cat-5e), a category 6 or 6a (Cat-6 or Cat-6a), a category 7 (Cat-7) cable, or some similar network cable, and transceiver 130 is an Ethernet transceiver. In still other embodiments, transceiver 130 may be constructed to include the functionality of the communication modules 56 disclosed in commonly assigned U.S. patent application Ser. No. 15/831,466 filed Dec. 5, 2017, by inventor Michael Hayes et al. and entitled NETWORK COMMUNICATION FOR PATIENT SUPPORT APPARATUSES, the complete disclosure of which is incorporated herein by reference.

Regardless of the specific structure included with transceiver 130, controller 100 is able to communicate with the local area network 134 (FIG. 5) of a healthcare facility in which the patient temperature management device 46 is positioned. When transceiver 130 is a wireless transceiver, it communicates with local area network 134 via one or more wireless access points 132. When transceiver 130 is a wired transceiver, it communicates directly via a cable coupled between patient temperature management device 46 and a network outlet positioned within the room of the healthcare facility in which patient temperature management device 46 is positioned.

Local area network 134 typically includes a plurality of servers, the contents of which will vary from healthcare facility to healthcare facility. In general, however, most healthcare facilities will include, among other servers, an electronic medical records (EMR) server 136, which may be a conventional server. In addition to EMR server 136, local area network 134 may include a patient support apparatus server 138 that is in communication with one or more patient support apparatuses 20 and/or patient temperature management devices 46 positioned within the healthcare facility. Server 138 may also be communicatively coupled (via the Internet or other means) to one or more other servers that are positioned outside of the healthcare facility. Further details regarding network 134 and the servers that may be hosted thereon are discussed below with respect to FIG. 6.

Although not shown in FIG. 5, patient temperature management device 46 may include a clock/calendar (not shown) that communicates with controller 100. The clock/calendar not only measures the passage of time, but it also keeps track of the calendar day (and year). The clock/calendar may be any conventional timing device that is able to keep track of the passage of time, including the calendar day and year.

In addition to the patient temperature sensor(s) 87, the water temperature sensor 98, and the reservoir temperature sensor 124 (if included), patient temperature management device 46 may include still more sensors that are positioned within main body 58, and/or that are positioned outside of main body 58 and in communication with main controller 100. Such off-board sensors (e.g. outside of main body 58) may communicate with main controller 100 via one or more of the auxiliary sensor ports 68 and/or via one or more of the transceivers 130. Each auxiliary sensor port 68 is adapted to receive outputs from an off-board auxiliary sensor 140. The auxiliary sensors 140, as well as any additional sensors onboard patient temperature management device 46, provide additional data to controller 100 regarding the patient during a thermal therapy session. Controller 100 is configured to utilize the additional data either for use in one or more algorithms that are currently being used by patient temperature management device 46 to control the patient's temperature, or for potential future use in one or more improved algorithms that are determined, after analysis, to provide improved results for the thermal therapy sessions carried out using patient temperature management device 46.

Auxiliary ports 68 (FIGS. 4 & 5) may take on a variety of different forms. In one embodiment, all of the ports 68 (if there are more than one) are of the same type. In another embodiment, patient temperature management device 46 includes multiple types of ports. In any of these embodiments, the ports 68 may include, but are not limited to, a Universal Serial Bus (USB) port, an Ethernet port (e.g. an 8P8C modular connector port, or the like), a parallel port, a different (from USB) type of serial port, etc. Ports 68 may also or alternatively be implemented wirelessly, such as via a WiFi transceiver, a Bluetooth transceiver, a ZigBee transceiver, etc.

Patient temperature management device 46 may be configured to accept a number of different types of auxiliary sensors 140 via input ports 68. Such sensors include, but are not limited to, the sensors 128 disclosed in commonly assigned PCT patent application serial number PCT/US2021/061947 filed Dec. 6, 2020, by applicant Stryker Corporation and entitled THERMAL CONTROL SYSTEMS WITH DYNAMIC CONTROL ALGORITHMS, the complete disclosure of which is incorporated herein by reference. In some embodiments, patient temperature management device 46 omits auxiliary ports 68 and is not configured to accept any auxiliary sensors 140.

FIG. 6 illustrates patient support apparatus 20 and thermal control system 48 positioned within a room of a healthcare facility. FIG. 6 also illustrates several additional items that may be present in a healthcare facility and which patient support apparatus 20 and/or patient temperature management device 46 are configured to communicate with, including, but not limited to, a locator unit 142 and a conventional local area network 134 of the healthcare facility. Locator units 142 are positioned at known and fixed locations within the healthcare facility in which patient support apparatus 20 are typically positioned. Locator units 142 function as fixed locators. That is, locator units 142 communicate with patient support apparatuses 20 and share information with them that allows the location of the patient support apparatuses 20 to be determined.

In some embodiments, patient support apparatus 20 is configured to be able to communicate with at least two different types of units: linked locator units and unlinked locator units. The linked and unlinked locator units 142 differ from each other in that the linked locator units are adapted to communicate with a conventional communication outlet 144 that is typically built into one or more walls of a healthcare facility. That is, the linked locator units 142 are communicatively linked to a conventional communication outlet 144. The unlinked locator units 142 are not adapted to communicate with such communication outlets 144, and are therefore not linked to a nearby communications outlet 144. Both types of locator units are adapted to provide location information to patient support apparatus 20. The linked locator units 142, however, are also adapted to serve as a communication conduit for routing communications between patient support apparatus 20 and one or more devices and/or systems that are communicatively coupled to communication outlet 144 (e.g. a reading light 146, a room light 148, a television 150, and/or a nurse call system 152 (FIG. 6)). The unlinked locator units 142, in contrast, are not adapted to serve as communication conduits between patient support apparatus 20 and these other devices and/or systems. In general, the linked locator units 142 are typically positioned in patient rooms of the healthcare facility where one or more communication outlets 144 are typically present, while the unlinked locator units 142 are typically positioned in locations outside of patient rooms, such as hallways, maintenance areas, and/or other areas. Unless explicitly stated otherwise, references herein to “locator units 142” refer to both types of locator units. Locator units 142 are adapted to enable the location of patient support apparatus 20 to be determined.

As shown in FIG. 6, linked locator units 142 are adapted to be mounted to a wall 154, such as a headwall of a patient room within the healthcare facility. The headwall of a conventional healthcare facility room typically includes a conventional communications outlet 144 physically integrated therein. Communications outlet 144 is adapted to receive a nurse call cable 156 that physically connects at its other end either to patient support apparatus 20 (not shown) or to locator unit 142 (shown in FIG. 6). In many healthcare facilities, communication outlet 144 includes a 37-pin connector, although other types of connectors are often found in certain healthcare facilities. As will be discussed in greater detail below, linked locator unit 142 and nurse call cable 156 allow patient support apparatus 20 to communicate with a nurse call system, and one or more room devices positioned within room.

Communication outlet 144 is electrically coupled to one or more cables, wires, or other conductors 158 that electrically couple the communication outlet 144 to a nurse call system 152 and one or more conventional room devices, such as a reading light 146, a room light 148, and/or a television 150. Conductors 158 are typically located behind the wall of the room and not visible to people in the room. In some healthcare facilities, conductors 158 may first couple to a room interface circuit board that includes one or more conductors 158 for electrically coupling the room interface circuit board to room devices 146, 148, 150 and/or nurse call system 152. Still other communicative arrangements for coupling communication outlet 144 to nurse call system 152 and/or one or more room devices 146, 148, and 150 are possible.

Nurse call cable 156 (FIG. 6) enables locator unit 142 to communicate with nurse call system 152 and/or room devices 146, 148, 150, and because patient support apparatus 20 is able to wirelessly communicate with locator unit 142, patient support apparatus 20 is thereby able to communicate with nurse call system 152 and room devices 146, 148, 150. A patient supported on patient support apparatus 20 who activates a nurse call control (e.g. 50g; see FIG. 3) on patient support apparatus 20 causes a signal to be wirelessly sent from patient support apparatus 20 to locator unit 142, which in turn conveys the signal via nurse call cable 156 to the nurse call system 152, which forwards the signal to one or more remotely located nurses (e.g. nurses at one or more nurse's stations 174). If the patient activates one or more room device controls (e.g. controls 50l-t; see FIG. 3), one or more wireless signals are conveyed to the locator unit 142, which in turn sends appropriate signals via nurse call cable 156 to communication outlet 144 and the room device 146, 148, 150 that change one or more features of these devices (e.g. the volume, channel, on/off state, etc.).

As is also shown in FIG. 6, patient support apparatus 20 is further configured to communicate with a local area network 134 of the healthcare facility. In the embodiment shown in FIG. 6, patient support apparatus 20 includes a wireless network transceiver 160 (FIG. 7) that communicates wirelessly with local area network 134. Network transceiver 160 is, in at least some embodiments, a WiFi transceiver (e.g. IEEE 802.11) that wirelessly communicates with one or more conventional wireless access points 132 of local area network 134. In other embodiments, network transceiver 160 may be a wireless transceiver that uses conventional 5G technology to communicate with network 134, one or more servers hosted thereon, and/or other devices. In some embodiments, network transceiver 160 may include any of the structures and/or functionality of the communication modules 56 disclosed in commonly assigned U.S. Pat. No. 10,500,401 issued to Michael Hayes and entitled NETWORK COMMUNICATION FOR PATIENT SUPPORT APPARATUSES, the complete disclosure of which is incorporated herein by reference. Still other types of wireless network transceivers may be utilized.

In some embodiments, network transceiver 160 is a wired transceiver that is adapted to allow patient support apparatus 20 to communicate with network 134 via a wired connection, such as an Ethernet cable that plugs into an Ethernet port (e.g. an RJ-45 style port, an 8P8C port, etc.) built into patient support apparatus 20. In still other embodiments, patient support apparatus 20 includes both a wired transceiver 160 for communicating with network 134 via a wired connection and a wireless transceiver 160 for wirelessly communicating with network 134.

Patient support apparatus 20 is configured to communicate with one or more servers on local area network 134 of the healthcare facility. One such server is a patient support apparatus server 138. Patient support apparatus server 138 is adapted, in at least one embodiment, to receive status information from patient support apparatuses 20 positioned within the healthcare facility and distribute this status information to caregivers, other servers, and/or other software applications. As will be discussed in greater detail below, server 138 may also be configured to receive data from one or more medical devices that are associated with patient support apparatus 20. Alternatively, in some embodiments where data from medical devices is collected, the data from one or more of medical devices may be forwarded by server 138 to one or more electronic devices 162 and/or to one or more other servers on network 134, such as a caregiver assistance server and/or a caregiver assistance software application, as will also be discussed in greater detail below.

In some embodiments, patient support apparatus server 138 is configured to communicate at least some of the patient support apparatus status data and/or medical device data received from patient support apparatuses 20 to a remote server 164 that is positioned geographically remotely from the healthcare facility. Such communication may take place via a conventional network appliance 166, such as, but not limited to, a router and/or a gateway, that is coupled to the Internet 168. The remote server 164, in turn, is also coupled to the Internet 168, and patient support apparatus server 138 is provided with the URL and/or other information necessary to communicate with remote server 164 via the Internet connection between network 134 and server 164.

In some alternative embodiments, patient support apparatus 20 may be configured to communicate directly with one or more cloud-based servers, such as remote server 164, without utilizing patient support apparatus server 138. That is, in some embodiments, patient support apparatuses 20 may be configured to communicate directly with a remote server without relying upon any locally hosted servers (e.g. servers hosted on network 134). In one such embodiment, patient support apparatus 20 utilizes Microsoft's Azure could computing service to directly connect to one or more remote servers 86 without utilizing server 138. In some such embodiments, network appliance 166 is a router configured to support such direct connections. Still other types of direct-to-cloud connections may be utilized with one or more of patient support apparatuses 20.

Patient support apparatus server 138 is also configured to determine the location of each patient support apparatus 20, or receive the location of each patient support apparatus 20 from the patient support apparatuses 20. In some embodiments, patient support apparatus server 138 determines the room number and/or bay area of each patient support apparatus 20 that is positioned within a room, as well as the location of patient support apparatuses 20 that are positioned outside of a room, such as those that may be positioned in a hallway, a maintenance area, or some other area. In general, patient support apparatus server 138 may be configured to determine the position of any patient support apparatus 20 that is positioned within communication range of one or more locator units 142, as will be discussed in greater detail below.

EMR server 136 (FIG. 6) is a conventional server that stores the patients' electronic medical records. Such electronic medical records typically contain medical information about a patient, such as the patient's treatment, medical history, prescriptions, and/or therapies, assessments, etc. Admissions, Discharge, and Transfer (ADT) server 170, which may be a part of EMR server 136 or a separate server, is a conventional server that keeps track of patients' room assignments and, in some versions, the caregivers assigned to the patients. ADT server 170 is used for managing the admission, discharge, and transfer of patients in the healthcare facility. The ADT server 170 stores patient location information, including the identity of patients and the corresponding rooms (and bay numbers in rooms with more than one patient). Patient support apparatus server 138 is adapted to communicate with both EMR serer 126 and ADT server 170.

In some embodiments, patient support apparatus server 138 (FIG. 6) is adapted to determine which patient is assigned to a particular room by communicating with ADT server. Additionally, patient support apparatus server 138 is adapted to store, or have access to, a table that lists the locations of each fixed locator unit 142 within the healthcare facility. When a patient support apparatus 20 reports the unique ID of the fixed locator 142 that it is currently associated with to patient support apparatus server 138, patient support apparatus server 138 uses this fixed locator ID to determine in which room or bed bay the patient support apparatus 20 is currently located. From this room number and/or bed bay number, patient support apparatus server 138 is adapted to determine which patient is assigned to that particular patient support apparatus 20 by requesting from ADT server 170 the identity of the patient who is currently assigned to that particular room and/or bay number. Once patient support apparatus server 138 determines which patient is assigned to a particular patient support apparatus 20, it is adapted to forward medical data that is generated from that patient support apparatus 20 to the appropriate patient record in EMR server 136. Similarly, any medical data that comes from a medical device that is currently associated with patient support apparatus 20 and that forwards its data to patient support apparatus server 138 is forwarded by patient support apparatus server 138 to EMR server 136.

The forwarding of medical data from a medical device associated with a particular patient support apparatus 20 can occur in at least two different manners. In a first manner, the medical device forwards its medical device to the associated patient support apparatus 20 directly via a Bluetooth transceiver, an ultra-wideband transceiver, or another transceiver, as will be discussed in more detail below. After receiving this medical data, patient support apparatus 20 forwards the medical data to patient support apparatus server 138 via its network transceiver 160. In a second manner, a medical device may include its own network transceiver and forward its medical data to patient support apparatus server 138 without routing it through a patient support apparatus 20. Regardless of how the medical data gets to patient support apparatus server 138, patient support apparatus server 138 automatically forwards the data to the correct patient's record in the EMR server 170.

The forwarding of patient support apparatus data and/or medical data from an associated medical device to the correct patient record in EMR server 136 takes place automatically. That is, it does not require a caregiver to take any manual steps to associate patient support apparatus 20 with a particular patient and/or to associate any of the medical devices discussed herein with patient support apparatus 20 (or a particular patient). Instead, these associations—as well as the automatic routing of data to the correct patient EMR record—are carried out automatically by patient support apparatus 20 and patient support apparatus server 136 after patient support apparatus 20 is moved to a location within a relatively small distance of a fixed locator (e.g. 1-3 meters or so). Once in this position, patient support apparatus 20 automatically reads that fixed locator's ID, associates itself with that fixed locator, and forwards its own unique ID and the fixed locator's ID to patient support apparatus server 138 via network transceiver 160. Patient support apparatus server 138 uses the fixed locator's ID to determine the patient support apparatus 20's room and/or bay number, and then uses that room number and/or bay number to determine the patient assigned to that particular room and/or bay number (by consulting ADT server 170). In addition, patient support apparatus server 138 thereafter automatically forwards medical data from that particular patient support apparatus 20 to the correct patient record in the EMR server 136. As will be discussed in greater detail, this all happens automatically once the patient support apparatus is moved within a threshold proximity to the fixed locator.

In addition, as will also be discussed in more detail below, patient support apparatus 20 is adapted to automatically associate one or more medical devices with itself and to share those associations with patient support apparatus server 138. Data from those medical devices is then automatically forwarded to patient support apparatus server 138, which then forwards it automatically to the correct EMR record. This automatic EMR data entry occurs for the associated medical devices regardless of whether those devices forward their data to patient support apparatus 20, which then forwards it to patient support apparatus server 138, or they forward their data to patient support apparatus server 138 without sending it to patient support apparatus 20. In other words, the route the data takes from the associated medical devices to patient support apparatus server 138 does not matter.

In an alternative embodiment, patient support apparatus server 138 is adapted to forward the patient ID associated with a particular patient support apparatus 20 to the patient support apparatus 20 and/or to the associated medical devices. In this embodiment, the patient support apparatus 20 and/or the associated medical devices can forward their medical data directly to EMR server 136 and bypass patient support apparatus server 138 because these devices are able to inform EMR server 136 of the corresponding patient record that such data is to be saved in. In other words, once the associated medical device and/or patient support apparatus 20 is informed of the patient to which they are associated, they are able to forward their data to EMR server 136 without passing through patient support apparatus server 138.

It will be understood that the architecture and content of local area network 134 will vary from healthcare facility to healthcare facility, and that the example shown in FIG. 6 is merely one example of the type of network a healthcare facility may be employ. Typically, one or more additional servers 172 will be hosted on network 134 and one or more of them may be adapted to communicate with patient support apparatus server 138. Local area network 134 will also typically allow one or more electronic devices 162 to access the local area network 134 via wireless access points 132. Such electronic devices 162 include, but are not limited to, smart phones, tablet computers, portable laptops, desktop computers, smart televisions, and other types of electronic devices that include a WiFi capability and that are provided with the proper credentials (e.g. SSID, password, etc.) to access network 134 (and, in at least some situations, patient support apparatus server 138).

Linked locator units 142 (FIG. 6) are adapted to wirelessly receive signals from patient support apparatus 20 and deliver the signals to communications outlet 144 in a manner that matches the way the signals would otherwise be delivered to communications outlet 144 if a conventional nurse call cable 156 were connected directly between patient support apparatus 20 and communications outlet 144. Linked locator units 142 are also adapted to transmit signals received from communications outlet 144 to patient support apparatus 20 via a BT transceiver 198 and/or a UWB transceiver 186 (FIG. 7). Thus, patient support apparatus 20 and linked locator unit 142 cooperate to send signals to, and receive signals from, communications outlet 144 in a manner that is transparent to communications outlet 144 such that outlet 144 cannot detect whether it is in communication with patient support apparatus 20 via a wired connection or it is in communication with patient support apparatus 20 via a wireless connection between patient support apparatus 20 and linked locator unit 142 (the latter of which is in wired communication with outlet 144). In this manner, a healthcare facility can utilize the wireless communication abilities of one or more patient support apparatuses 20 without having to make any changes to their existing communication outlets 144.

As noted, in addition to sending signals received from patient support apparatus 20 to communications outlet 144, linked locator units 142 are also adapted to forward signals received from communications outlet 144 to patient support apparatus 20. Linked locator units 142 are therefore adapted to provide bidirectional communication between patient support apparatus 20 and communications outlet 144. This bidirectional communication includes, but is not limited to, communicating command signals from any of controls 50 to corresponding room devices 146, 148, and/or 150 and communicating audio signals between a person supported on patient support apparatus 20 and a caregiver positioned remotely from patient support apparatus 20. The audio signals received by locator units 142 from a microphone on patient support apparatus 20 are forwarded to communications outlet 144 (for forwarding to nurse call system 152), and the audio signals of a remotely positioned nurse that are received at communications outlet 144 (from nurse call system 152) are forwarded to a speaker onboard patient support apparatus 20.

Nurse call cable 156, in some embodiments, includes a conventional 37 pin connector on each end, one of which is adapted to be inserted into outlet 144 and the other one of which is adapted to be inserted into locator unit 142. Such 37 pin connections are one of the most common types of connectors found on existing walls of medical facilities for making connections to the nurse call system 152 and room devices 146, 148, and 150. Linked locator unit 142 and nurse call cable 156 are therefore configured to mate with one of the most common type of communication outlets 144 used in medical facilities. Such 37 pin connectors, however, are not the only type of connectors, and it will be understood that linked locator units 142 can utilize different types of connectors that are adapted to electrically couple to different types of nurse call cables 156 and/or different types of communication outlets 144. One example of such an alternative communications outlet 144 and cable 156 is disclosed in commonly assigned U.S. patent application Ser. No. 14/819,844 filed Aug. 6, 2015 by inventors Krishna Bhimavarapu et al. and entitled PATIENT SUPPORT APPARATUSES WITH WIRELESS HEADWALL COMMUNICATION, the complete disclosure of which is incorporated herein by reference. Still other types of communication outlets 144 and corresponding connectors may be utilized.

Linked locator unit 142 (FIG. 6) also includes an electrical cord 176 having a plug positioned at a far end that is adapted to be inserted into a conventional electrical outlet 178. Electrical cord 176 enables linked locator unit 142 to receive power from the mains electrical supply via outlet 178. It will be appreciated that, in some embodiments, linked locator unit 142 is battery operated and cord 176 may be omitted. In still other embodiments, linked locator unit 142 may be both battery operated and include cord 176 so that in the event of a power failure, battery power supplies power to linked locator unit 142, and/or in the event of a battery failure, electrical power is received through outlet 178. Unlinked locator units 142 may also include a battery, electrical cord, or both.

In some embodiments, locator units 142 (linked and/or unlinked) include a video port that is adapted to receive a display cable 180 (FIG. 6). The display cable 180 is adapted to couple to locator unit 142 at one end and a display device 182 at its opposite send. Locator unit 142 is configured to use cable 180 to send data to display device 182 that is to be displayed thereon. Such data may include data from patient temperature management device 46, data from one or more vital sign sensors that are coupled to the patient on patient support apparatus 20, status data from one or more sensors onboard patient support apparatus 20, location data regarding the location of patient support apparatus 20, and/or other data. Some examples of the type of data that may be displayed on display device 182 are shown in FIG. 9. Cable 180 may be a High-Definition Multimedia Interface (HDMI) cable, a Video Graphics Array (VGA) cable, a DisplayPort (DP) cable, a plurality of Radio Corporation of America (RCA) cables, a Digital Visual Interface (DVI) cable, and/or another type of cable. Locator unit 142 is configured to include a complementary type of connector that mates with a connector on an end of cable 180. Patient support apparatus 20 may also, or alternatively, be configured to communicate directly with certain display devices without using locator unit 142 as a communication intermediary.

In addition to any of the structures and functions described herein, locator units 142 are configured to communicate location data to patient support apparatus 20 that enables patient support apparatus 20 and/or patient support apparatus server 138 to determine the location of patient support apparatus 20 within the healthcare facility. In general, such location determination is carried out by patient support apparatus 20 analyzing wireless signals communicated between itself and locator unit 142 in order for it to determine its position relative to locator unit 142. After determining its relative position to locator unit 142, patient support apparatus 20 is configured to determine if its relative position meets an association condition. If it does, patient support apparatus 20 uses the receipt of a unique wall identifier (ID) from locator unit 142 to determine, or have patient support apparatus server 138 determine, its absolute position within the healthcare facility. The location of each locator unit 142 in the healthcare facility is surveyed during the installation of locator units 142, and the unique IDs of each locator unit 142 are also recorded during the installation of locator units 142. This surveying information and corresponding ID information may be stored in patient support apparatus server 138 and/or onboard patient support apparatus 20, thereby enabling patient support apparatus 20 and/or patient support apparatus server 138 to determine the location of a patient support apparatus 20 once its relative position to an identified locator unit 142 is known.

If the location of patient support apparatus 20 is determined remotely, patient support apparatus 20 sends its relative position information and the ID of the locator unit 142 (and its own unique patient support apparatus ID 184 (FIG. 7) to server 138. Server 138 includes a table of all of the locations of the locator units 142 (which, as noted, is generated via a surveying operation during the installation of locator units 142), and it uses that table to correlate the patient support apparatus IDs 184 and the locator unit IDs it receives to specific locations within the healthcare facility. Thus, if a particular patient support apparatus 20 (with a particular ID 184) sends a locator unit ID that corresponds to room 430, server 138 determines that that particular patient support apparatus 20 is currently located in room 430. Generally speaking, and as will be discussed in greater detail below, the location of a patient support apparatus 20 is deemed to correspond to whichever locator unit 142 it is currently associated with, and if it is not currently associated with any locator unit 142, its location may be indeterminate.

In some embodiments of patient support apparatus 20 and locator unit 142, the relative location of patient support apparatus 20 to a locator unit 142 is carried out solely using ultra-wideband communication between the patient support apparatus 20 and the locator unit 142. Alternatively, in some embodiments, patient support apparatus 20 solely uses short range infrared communications with locator unit 142 to determine its relative location, wherein such short range infrared communications are only possible when the patient support apparatus 20 is positioned within a close proximity to the locator unit 142 (e.g. in the range of about 1-3 meters). In these latter embodiments, patient support apparatus 20 may report to server 138 that its location coincides with that of the nearby locator unit 142 when it is able to successfully communicate with the nearby locator unit 142 using these short range infrared communications, and to not report any location data when it is not able to successfully communicate with the nearby locator unit 142. Still further, in some embodiments, patient support apparatus 20 and locator units 142 may communicate with each other using both infrared and ultra-wideband communications. Further details regarding the use of short range infrared communications for location determination are described in commonly assigned U.S. Pat. No. 9,999,375 issued Jun. 19, 2018, to inventors Michael Hayes et al. and entitled LOCATION DETECTION SYSTEMS AND METHODS, the complete disclosure of which is incorporated herein by reference.

In some embodiments, locator units 142 and/or patient support apparatuses 20 may be constructed to include any or all of the functionality of the wireless headwall units and/or patient support apparatuses disclosed in commonly assigned U.S. patent application Ser. No. 14/819,844 filed Aug. 6, 2015, by inventors Krishna Bhimavarapu et al. and entitled PATIENT SUPPORT APPARATUSES WITH WIRELESS HEADWALL COMMUNICATION; in commonly assigned U.S. patent application Ser. No. 63/26,937 filed May 19, 2020, by inventors Alexander Bodurka et al. and entitled PATIENT SUPPORT APPARATUSES WITH HEADWALL COMMUNICATION; and/or in commonly assigned U.S. patent application Ser. No. 63/245,245 filed Sep. 17, 2021, by inventors Kirby Neihouser et al. and entitled SYSTEM FOR LOCATING PATIENT SUPPORT APPARATUSES, the complete disclosures of all of which are incorporated herein by reference.

Still further, in some embodiments, locator units 142 and/or patient support apparatuses 20 may be constructed to include any of the features and/or functions of the headwall units 144a and/or patient support apparatuses disclosed in commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which is incorporated herein by reference.

FIG. 7 depicts a block diagram of patient support apparatus 20, a linked locator unit 142, a patient temperature management device 46, a thermal wrap 54, a hose 56, and a display device 182. As will be discussed in greater detail below, patient support apparatus 20 is configured to automatically determine whether to associate the patient temperature management device 46 with itself using position information regarding patient temperature management device 46, hose 56, and thermal wrap 54. Although not shown in FIG. 7, patient support apparatus 20 may also be configured to determine whether to associate other medical devices, such as vital sign sensors and other sensors, with itself using position information regarding those other device. As will be discussed in greater detail below, patient support apparatus 20 uses the position information of these devices and one or more predefined association conditions that need to be met in order to determine whether to associate itself with a particular device. In some embodiments, if patient support apparatus 20 associates itself with a particular device, it may send data from that device (or devices) to one or more of display devices 182, and/or it may forward data from that device (or devices) to patient support apparatus server 138 and/or EMR server 136.

Locator units 142 (both linked and unlinked) include an ultra-wideband transceiver 186, a locator unit controller 188, and a unit ID 190 (FIG. 7). Linked locator units 142 further include configuration circuitry 192, a television controller 194, a headwall interface 196, and a Bluetooth transceiver 198 (FIG. 7). In some embodiments, linked and/or unlinked locator units 142 may further include a video port 200 and/or an infrared transceiver 202. Bluetooth transceiver 198 is adapted to communicate with a Bluetooth transceiver 210 onboard patient support apparatus 20 using RF waves in accordance with the conventional Bluetooth standard (e.g. IEEE 802.14.1 and/or the standard maintained by the Bluetooth Special Interest Group (SIG) of Kirkland, Washington, USA). In some embodiments, transceivers 198 and 210 utilize Bluetooth Low Energy communications.

Ultra-wideband transceiver 186 is adapted to communicate with one or more ultra-wideband transceivers 212 positioned onboard patient support apparatus 20. Transceiver 186 is adapted to determine a distance between itself and patient support apparatus 20. Alternatively, or additionally, transceiver 186 may be adapted to allow each transceiver 212 onboard patient support apparatus 20 to determine its distance from transceiver 186. In some embodiments, transceivers 186 and 212 use time of flight (TOF) computations to determine these distances. In other embodiments, transceiver 186 and 212 may utilize other techniques for determining their distances from each other, either in addition to, or in lieu of, TOF computations. In some embodiments, transceivers 186, 212 may also determine an angle between themselves using angular information derived from antenna arrays positions onboard transceivers 186, 212, or by using other techniques. In some embodiments, as will be discussed more below, three or more UWB transceivers 212 are positioned onboard patient support apparatus 20 and determine the relative position of UWB transceiver 186 by measuring the time difference of arrival at each transceiver 212 of a UWB signal sent from UWB transceiver 186. These time differences of arrival are used with the position and orientation of each transceiver 212 onboard patient support apparatus 20, which are known and stored in an onboard memory, to determine the position and orientation of patient support apparatus 20 with respect to the locator unit(s) 142. Such position and/or orientation determinations may be carried out using conventional triangulation and/or trilateration techniques using the distance measurements and the known positions and orientations of UWB transceivers 212 (and in some cases the angle measurements may also be used as well).

In some embodiments, transceivers 186, 212 (FIG. 7) are implemented as any of the Trimension™ ultra-wideband modules available from NXP Semiconductors of Austin, Texas. These modules include, but are not limited to, the Trimension™ UWB modules ASMOP1BO0N1, ASMOP1CO0R1, and/or the ASMOP1CO0A1, that utilize any of the following chips: the NXP SR150, SR100T, SR040, NCJ29D5, and/or the OL23DO chips. Modules manufactured and/or marketed by other companies may also be used, including, but not limited to, the Decawave DWM1000, DWM10001C, DWM3000 modules (available from Decawave of Dublin, Ireland); the Nordic TSG5162 SiP module (available from Tsingoal Technology of Beijing, China); and/or the UWB hub, wand, and/or sensors available from Zebra technologies of Lincolnshire, Illinois. Still other types of UWB modules may be used to implement transceivers 186 and 124.

Locator unit controller 188 is adapted to control the operation of transceivers 186, 198, configuration circuitry 192, TV controller 194, headwall interface 196, video port 200, and, if included, IR transceiver 202 (FIG. 7). When infrared transceiver 202 is included, it may be included to provide backwards compatibility to patient support apparatuses 20 that are not equipped with a UWB transceiver 212. That is, some healthcare facilities may include one or more patient support apparatuses that are not equipped with a UWB transceiver 212, but that do include an IR transceiver that is adapted to communicate with IR transceiver 202. When linked locator unit 142 includes IR transceiver 202, it is able to communicate its unit ID 190 to such patient support apparatuses via IR transceiver 202, which is a short range transceiver that is configured to only communicate with an adjacent patient support apparatus when the patient support apparatus is nearby (e.g. without about 1 to 3 meters or so). Such an adjacent patient support apparatus then communicates the received locator unit ID 190 along with its own unique ID 184 to server 138 which, as noted previously, is able to correlate the locator unit ID 190 to a particular location with the healthcare facility. In this manner, server 138 is able to use locator units 142 determine the location of versions of patient support apparatuses 20 that don't have a UWB transceiver 186, but that do have an IR transceiver.

Headwall interface 196 (FIG. 7) is adapted to change the electrical state of one or more pins that are in electrical communication with communication outlet 144 (via cable 156). Headwall interface 196 changes these electrical states in response to instructions from controller 188. For example, if the exit detection system 214 of patient support apparatus 20 detects a patient exit, a controller 216 of patient support apparatus 20 sends an exit alert signal to linked locator unit 142 and controller 188 responds by instructing headwall interface 196 to change the electrical state of at least one pin that is used to signal an exit alert (or a generic priority alert) to the nurse call system 152 via communications outlet 144. In some embodiments, headwall interface 196 may be constructed in the same manner as, and/or may include any one or of the functions as, the cable interface 88 described in commonly assigned U.S. patent application Ser. No. 63/193,778 filed May 27, 2021, by inventors Krishna Bhimavarapu et al. and entitled PATIENT SUPPORT APPARATUS AND HEADWALL UNIT SYNCING, the complete disclosure of which is incorporated herein by reference. Alternatively, or additionally, headwall interface 196 may be constructed in the same manner as, and/or may include any one or more of the same functions as, the headwall interface 120 disclosed in commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which is incorporated herein by reference. Linked locator unit 142 may also be configured to perform any of the functions of the headwall units 94 disclosed in the above-mentioned '778 patent application.

Configuration circuitry 192 and TV controller 194 may be configured to perform any of the same functions as, and/or be constructed in any of the same manners as, the configuration circuitry 132 and the TV control circuit 134, respectively, of commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which has already been incorporated herein by reference. Additionally, or alternatively, linked locator unit 142 may be configured to perform any of the functions of the headwall units 144 disclosed in the aforementioned '508 patent application.

Patient support apparatus 20 includes a controller 216, a memory 218, exit detection system 214, a microphone 220, Bluetooth transceiver 210, one or more UWB transceivers 212, display 52, network transceiver 160, and a plurality of additional components that are not shown in FIG. 7. Each UWB transceiver 212 is positioned at a known location on patient support apparatus 20. This known location information is stored in memory 218 and/or elsewhere, and may be defined with respect to any suitable common frame of reference. The known location information may include the spatial relationship between UWB transceivers 212 and/or any other components of patient support apparatus 20. For example, in some embodiments, the known location information includes the spatial relationship not only between UWB transceivers 212, but also the spatial relationships between UWB transceivers 212 and one or more of the following: the head end 38 of patient support apparatus 20, the foot end of patient support apparatus 20, the sides of patient support apparatus 20, the floor, and/or other components and/or landmarks of patient support apparatus 20. In some embodiments, this location information is used to determine the position and/or orientation of patient support apparatus 20 with respect to one or more walls, locator units 142, another patient support apparatus 20, and/or other objects or structures within the healthcare facility.

Controller 216, as well as controller 188, may take on a variety of different forms. In the illustrated embodiment, each of these controllers is implemented as a conventional microcontroller. However, these controllers may be modified to use a variety of other types of circuits-either alone or in combination with one or more microcontrollers-such as, but not limited to, any one or more microprocessors, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. The instructions followed by controllers 188 and 216 when carrying out the functions described herein, as well as the data necessary for carrying out these functions, are stored in a corresponding memory that is accessible to that particular controller (e.g. memory 218 for controller 216, and a memory (not shown) for controller 188). In some embodiments, controller 216 may include and/or work with a microcontroller that is integrated into, or associated with, UWB transceiver(s) 212, and controller 188 may include and/or work with a microcontroller that is integrated into, or associated with, UWB transceiver 186.

Controller 216 utilizes transceivers 212 to determine the relative position of patient support apparatus 20 with respect to nearby devices, including one or more nearby locator units 142. If patient support apparatus 20 is positioned within range of a locator unit 142, its UWB transceivers 212 communicate with the UWB transceiver 186 positioned on that locator unit 142, and the transceivers 212 and 186 exchange signals that enable them to determine the distance between themselves, the relative position of each other, and/or the orientation with respect to each other. As was noted, in some embodiments, patient support apparatus 20 includes at least three UWB transceivers 212, and patient support apparatus 20 determines the distance between, and/or relative position of, each one of these UWB transceiver 212 and UWB transceiver 186 of locator unit 142.

In some embodiments, UWB transceivers 186, 212 (FIG. 7) may also be configured to determine an angular relationship between themselves. The distance (and angle information) in at least some embodiments is calculated by UWB transceiver 212 and controller 216 of patient support apparatus 20. In other embodiments, UWB transceiver 186 and controller 188 may calculate the distance (and angle information) and forward the results of this calculation to patient support apparatus 20 (either via UWB transceiver 186 or BT transceiver 198). In either situation, patient support apparatus controller 216 is informed of the distances (and, in some embodiments, as noted, the angle information) between transceivers 212 and 186. These distances and orientations are then used to calculate a relative position of patient support apparatus 20 to the locator unit 142 in a common frame of reference that, as will be discussed in greater detail below, may be defined in a fixed relationship to the patient support apparatus 20 or in a fixed relationship to the locator unit 142.

The location of patient support apparatus 20 relative to locator units 142 is repetitively determined by an exchange of communication signals between UWB transceivers 186 and 212. This exchange is initiated by an interrogation signal that may be sent by the UWB transceivers 186 of the locator unit 142, and/or it may be sent by the UWB transceivers 212 of the patient support apparatuses 20. The trigger for sending these interrogation signals (from either source) may simply be the passage of a predefined interval of time, in at least some embodiments. That is, in some embodiments, patient support apparatus 20 and/or locator units 142 may be configured to periodically send out an interrogation signal that will be responded to by any UWB transceivers 186 or 212 that are positioned with range of that signal. In those embodiments where patient support apparatuses 20 are configured to send out such an interrogation signal, the time intervals between the interrogation signals may be varied depending upon the location and/or other status of the patient support apparatus 20. For example, in some embodiments, patient support apparatuses 20 may be configured to send out the interrogation signals with longer timer intervals between them when the patient support apparatus is stationary, and to send out the interrogation signals with shorter time intervals between them when the patient support apparatus 20 is in motion. The interrogation signals, in some embodiments, that are sent out by patient support apparatus 20 are also used to interrogate not only UWB transceiver 186 of locator unit 142, but also any UWB-equipped devices that are positioned within range of the UWB transceivers 212. Such UWB-equipped devices may include, for example, a patient temperature management device 46, one or more hoses 56 with UWB transceivers, one or more thermal wraps 54 with UWB transceivers, and/or one or more other types of medical devices.

The measured distances (and/or angular information) between UWB transceivers 212 and the UWB transceivers of the other in-range UWB-equipped devices may be generated using Angle of Arrival (AoA) information, Time of Flight (TOF) information, Channel State Information, Time Difference of Arrival (TDoA) information, Two-Way Ranging (TWR) ranging information, and/or other information that is generated from the communication between the UWB transceivers. In some embodiments, each UWB transceiver includes an array of antennas that are used to generate distance and/or angular information with respect to the UWB transceiver in which it is in communication. Still further, in some embodiments, UWB transceivers 212 include one or more of their own microcontrollers, and the location of the other UWB transceiver(s) on other devices may be determined by these internal microcontrollers without utilizing controller 216. In other embodiments, controller 216 may work in conjunction with a controller onboard the UWB-equipped device to determine their relative locations to each other.

Patient support apparatus 20 also includes, in at least some embodiments, a microphone 220 (FIG. 7) that is used to detect the voice of the patient when the patient wants to speak to a remotely positioned nurse. The patient's voice is converted to audio signals by microphone 220 and controller 216 is adapted to forward these audio signals to an adjacent communications outlet 144 positioned in wall 154 (FIG. 6). When a cable 156 is coupled between patient support apparatus 20 and outlet 144, controller 216 forwards these audio signals to outlet 144 via the cable. When no such cable 156 extends between patient support apparatus 20 and outlet 144, controller 216 wirelessly forwards these audio signals to the locator unit 142 that it is currently associated with (using transceiver 210, or in some embodiments, transceiver 212) and controller 188 of locator unit 142 forwards these audio signals to outlet 144. As was noted, outlet 144 is in electrical communication with a conventional nurse call system 152 that is adapted to route the audio signals to the correct nurse's station 78, and/or other location. In some embodiments, microphone 220 acts as both a microphone and a speaker. In other embodiments, a separate speaker may be included in order to communicate the voice signals received from the remotely positioned nurse. In some embodiments, the audio communication between patient support apparatus 20 and communications outlet 144 is carried out in any of the manners, and/or includes any of the structures, disclosed in commonly assigned U.S. patent application Ser. No. 16/847,753 filed Apr. 14, 2020, by inventors Alexander Bodurka et al. and entitled PATIENT SUPPORT APPARATUSES WITH NURSE CALL AUDIO MANAGEMENT, the complete disclosure of which is incorporated herein by reference.

After the installation of locator units 142 in a particular healthcare facility, the location of each locator unit 142 within that facility is recorded. In some embodiments, the coordinates of the locations of locator units 142 are recorded in a common frame of reference (or converted to a common frame of reference after recordation). Such coordinates may be three dimensional (i.e. include a height components), or they may be two dimensional (no height component). In other embodiments, a more generalized location of one or more locator units 142 is determined, rather than the precise coordinates of the locator units 142. In still other embodiments, the locations of one or more locator units 142 are determined both generally and more precisely. The generalized location of the locator units 142 may include an indication of the room, bay, area, hallway, portion of a hallway, wing, maintenance area, etc. that the locator unit 142 is positioned in. The specific location of the locator units 142, as noted, may include an X, Y, and Z coordinate within a common frame of reference.

Regardless of how the location of each locator unit 142 is initially determined after they are installed in a healthcare facility (e.g. whether their coordinates are determined or a more generalized location is determined), the locations of all of the locator units 142, as well as their unique IDs 190, are stored in a memory accessible to server 138. Server 138 then uses this location data and ID data to determine the location of a patient support apparatus 20. Alternatively, or additionally, the location data and ID data are forwarded to patient support apparatuses 20 for storage in their onboard memories and for use in determining their own locations. In some embodiments, the location of each locator unit 142 (whether specific and/or general) may also, or alternatively, be stored in a memory within that particular locator unit 142 and shared with the devices it communicates with (e.g. patient support apparatuses 20). In some other embodiments, the location of each locator unit 142 may be stored in multiple locations.

It will be appreciated that patient support apparatuses 20 are configured to communicate with locator units 142 regardless of the orientation of the patient support apparatus 20. That is, the UWB transceivers 186 and 212 are radio frequency transceivers that do not rely on line of sight communication, unlike the IR transceiver 202 (if present). Thus, the patient support apparatuses 20 do not have to be pointed in any particular direction with respect to the locator units in order for transceivers 186 and 212 to communicate. This differs from some prior art systems that use IR communication between the patient support apparatuses 20 and the locator units and that require the IR transceiver onboard the patient support apparatus to be aimed toward the locator unit in order for communication to be established. It will also be understood that locator units 142 can be positioned on wall, columns, ceilings, or any other fixed structures within the healthcare facility.

As noted, patient support apparatus 20 is also configured to use UWB transceivers 212 to determine the position of various other devices relative to patient support apparatus 20, such as one or more patient temperature management devices 46, one or more hoses 56, one or more thermal wraps 54, one or more vital sign sensors, one or more display devices 182, and/or one or more other types of devices. As will be discussed in greater detail below, controller 216 uses UWB transceivers 212 to determine the relative position of these devices by communicating with one or more respective UWB transceiver that are either built into those devices or attached to a tag that is affixed to those devices. Such UWB transceivers operate in the same manner as UWB transceivers 212 and/or UWB transceiver 186 of locator units 142. And, as will also be discussed in greater detail below, controller 216 of patient support apparatus 20 uses the relative position information to determine how it will interact with these devices, including whether to associate itself with these devices, whether to display data from these devices, and/or whether to send data from one or more of these devices to patient support apparatus server 138 and/or EMR server 136.

Each hose 56 includes two connectors 222 (FIG. 7). One connector 222 is positioned at a first end of hose 56 and adapted to releasably couple to a thermal wrap 54 and the other connector 222 is positioned at a second end of hose 56 and adapted to releasably couple to patient temperature management device 46. Although FIG. 7 only illustrates a single hose 56 and a single thermal wrap 54, it will be understood that patient temperature management device 46 is typically utilized with multiple hoses 56 coupled between itself and multiple thermal wraps 54. In the particular embodiment show in FIG. 1, six hoses 56 are coupled between patient temperature management device 46 and three thermal wraps 54. Other numbers of hoses 56 and thermal wraps 54 may, of course, be used.

At least one of the hoses 56 (and potentially more than one hose 56) includes a pair of UWB transceivers 224, one of which is positioned adjacent a first end of the hose 56 and the other of which is positioned adjacent the second end of the hose 56. In some embodiments, each UWB transceiver 224 is incorporated into a UWB tag that is affixed to the hose 56. In other embodiments, each UWB transceiver 224 may be integrated into the hose and/or into the adjacent connector 222. As will be discussed in greater detail below, UWB transceivers 224 are adapted to communicate with the UWB transceiver 118 onboard patient temperature management device 46 and a UWB transceiver 230 coupled to one or more of the thermal wraps 54. In some embodiments, transceivers 224 are also adapted to communicate with UWB transceivers 212 positioned onboard patient support apparatus 20 and/or onboard locator unit 142. As will also be discussed in more detail below, UWB transceivers 224 of hose 56 are adapted to communicate with, and determine their distance from, the UWB transceivers 118 and 230, respectively, positioned onboard patient temperature management device 46 and thermal wrap(s) 54.

Thermal wrap 54 (FIG. 7) includes UWB transceiver 230 and at least one hose port 232. Hose port 232 is adapted to couple to hose connector 222. In some embodiments, each thermal wrap 54 includes two hose ports 232: one of which is for coupling to a supply hose 56a and the other one of which is for coupling to a return hose 56b. As was noted, UWB transceiver 230 is adapted to communicate with the adjacent UWB transceiver 224 so that a distance D between transceiver 230 and 224 can be determined. If this distance is less than a threshold, controller 216 of patient support apparatus 20 is configured to conclude that hose 56 is coupled to one of ports 232. The threshold distance can vary from embodiment to embodiment based on the particular location of UWB transceiver 230 relative to hose ports 232, but generally is on the order of about half a meter.

In some embodiments, UWB transceiver 230 is integrated into a tag that is attached to thermal wrap 54. In other embodiments, UWB transceiver 230 may be built into thermal wrap 54. In either embodiment, a battery (not shown) may be included in order to provide electrical power to UWB transceiver 230. A battery may also be provided for each of UWB transceivers 224 in order to provide electrical power thereto.

Display device 182 includes a display 240 and a display controller 242. Display device 182 may take on a variety of different forms. In some embodiments, display device 182 may be a conventional smart phone, laptop computer, tablet computer, smart TV and/or smart monitor. Display device 182 may interact with patient support apparatus 20 in any of the same manners that the patient support apparatus and display devices interact with each other that are disclosed in commonly assigned U.S. patent application Ser. No. 63/306,279 filed Feb. 3, 2022, by inventors Madhu Thota et al. and entitled COMMUNICATION SYSTEM FOR PATIENT SUPPORT APPARATUS, the complete disclosure of which is incorporated herein by reference. That is, patient support apparatus 20 may be configured to automatically associate itself with one or more display devices 182 and forward data to be displayed thereon, as will be described in more detail below. Additionally, or alternatively, display device 182 may be a mobile display device that is not coupled to fixed locator 142, and/or it may be a stationary display device that includes its own UWB transceiver that is used for association purposes with patient support apparatus 20.

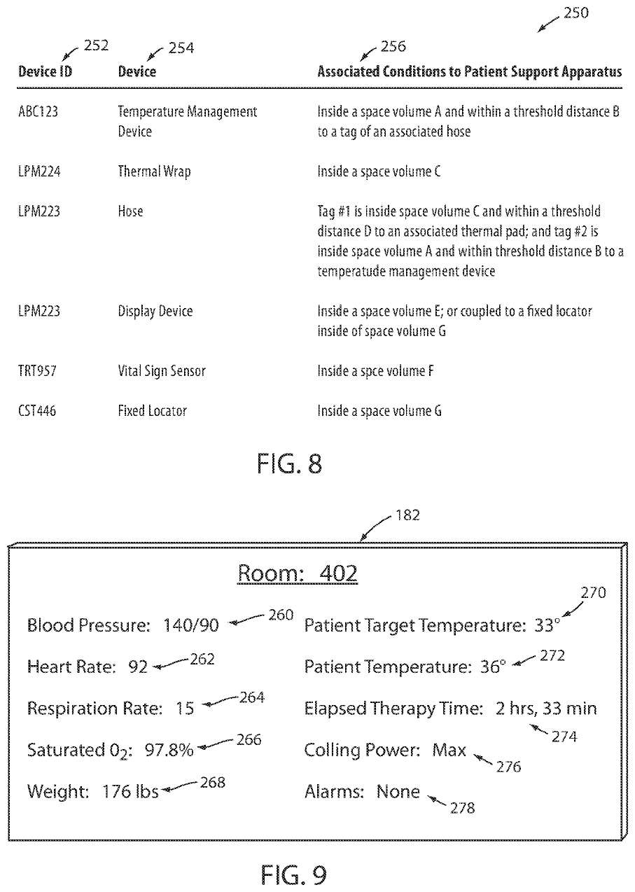

FIG. 8 illustrates a logic table 250 that patient support apparatus 20, in some embodiments, is configured to use when determining whether to associate one or more devices with itself. Logic table 250 includes a first column 252, a second column 254, and a third column 256. First column 252 lists a plurality of device IDs. Second column 254 lists the identity of the devices having the corresponding IDs. And third column 256 lists the condition(s) that must be satisfied before controller 216 of patient support apparatus 20 will consider the corresponding device to be associated with patient support apparatus 20. All or a portion of logic table 250 may be stored within memory 218 of patient support apparatus 20. Alternatively, all or a portion of logic table 250 may be stored within a memory accessible to patient support apparatus server 138. As yet another alternative, each device listed within table 250 may store the data in its corresponding row of table 250 and forward that data to patient support apparatus 20 when it is within communication range.

The device IDs of first column 252 are forwarded to patient support apparatus 20 via the UWB transceiver positioned onboard that particular device. For example, when a thermal wrap 54 is positioned within communication range of UWB transceivers 212 of patient support apparatus 20, UWB transceiver 230 of thermal wrap 54 forwards the corresponding device ID (LPM224 in the example shown in FIG. 8) of thermal wrap 54 to controller 216 of patient support apparatus 20. The device IDs shown in first column 252 uniquely identify each device. Further, in some embodiments, the device IDS of first column 252 distinguish the devices from other devices of the same type (e.g. a first thermal wrap 54 may have a different device ID than a second thermal wrap 54). The device ID in first column 252 for patient temperature management device 46 corresponds to unique ID 126, and the device ID in first column 252 for fixed locator unit 142 corresponds to unique ID 190. In some embodiments, both of the UWB transceivers 224 that are attached to a particular hose 56 transmit the same device ID to patient support apparatus 20 so that controller 216 is able to know that these UWB transceivers 224 are attached to a common hose 56.

When controller 216 receives a particular device ID from a device that is positioned within communication range of UWB transceivers 212, it consults the association conditions of column 256 (FIG. 8) to determine whether to associate that device with patient support apparatus 20 or not. This process takes place repeatedly while the device is positioned within communication range. The frequency of this repetition may vary from embodiment to embodiment, and/or from device to device, but generally may range from multiple times a second to once every several seconds and/or several minutes.

One example of the association conditions for a patient temperature management device 46 is shown in the first row of third column 256 of logic table 250 (FIG. 8). There are two association conditions shown therein for patient temperature management device 46: (1) the patient temperature management device 46 must be positioned within a volume of space A that is defined around patient support apparatus 20, and (2) the patient temperature management device 46 must also be within a threshold distance B to a UWB transceiver 224 of an associated hose. Turning to the first association condition, FIG. 6 illustrates an example of a volume of space A. As can be seen therein, volume A is generally centered about the center of patient support apparatus 20 and extends outward therefrom in all directions. Although FIG. 6 illustrates volume of space A having generally rectangular sides, it will be understood that this is merely for illustrative purposes, and that the shape, dimensions, and position of volume of space A can vary from what is shown in FIG. 6.

In general, volume of space A has a size that is large enough to encompass the space in which a patient temperature management device 46 would be positioned if the device 46 were being used on a patient supported on patient support apparatus 20. The size of volume of space A is therefore related to the length of hoses 56 that are typically used at a given healthcare facility. If a facility typically uses hoses 56 that are three meters long, for example, then volume of space A should extend outward horizontally from patient support apparatus 20 approximately three meters or more so that any patient temperature management device 46 that was used with a patient on patient support apparatus 20 would have to be positioned within volume of space A (assuming hoses 56 were used that were not longer than the typical length). The size of volume of space A should generally not be much larger than the length of the hoses 56 used within a given healthcare facility in order to reduce the possibility of other patient temperature management devices 46 being positioned with volume of space A that are being used to treat other patients who are not assigned to patient support apparatus 20. However, as will be discussed in greater detail below, if another patient temperature management device 46 is positioned with volume of space A, patient support apparatus 20 is configured to automatically determine which of the multiple patient temperature management devices 46 is the one being used with the patient assigned to itself, and to therefore automatically associate itself with only the correct one of the multiple patient temperature management devices 46.

Turning to the second association condition of patient temperature management device 46 (FIG. 8), this requires that one end of a hose 56 is positioned within a threshold distance B of one or more of the outlet ports 62 and/or inlet ports 64 of the patient temperature management device 46. An example of this threshold distance B is shown in FIG. 7. This distance is determined by UWB communication between UWB transceiver 224 of hose 56 and UWB transceiver 118 of patient temperature management device 46. The threshold B is chosen so that it is generally not met if hose 56 does not have its connector 222 connected to one or more of the outlet ports 62 and/or inlet ports 64 of the patient temperature management device 46. As was noted previously, the exact value of threshold distance B may vary in accordance with the position of UWB transceiver 118 relative to outlet ports 62 and/or inlet ports 64 of the patient temperature management device 46, and/or in accordance with the position of UWB transceiver 224 relative to connector 222. In general, the closer UWB transceiver 118 is positioned to one or more of ports 62 and/or 64, and the closer UWB transceiver 224 is positioned to connector 222, the smaller the value of threshold distance B, and vice versa.

Part of the second association condition of patient temperature management device 46 is that, not only must an end of hose 56 be positioned within a threshold distance B of a port 62 and/or 64 of patient temperature management device 46, but the hose 56 must be an associated hose. That is, patient support apparatus 20 must first determine whether the hose 56 is associated with patient support apparatus 20 before it can ultimately decide on whether to associate patient temperature management device 46 with itself or not.

Returning to the logic table 250 of FIG. 8, it can be seen that controller 216 of patient support apparatus 20 uses four criteria (association conditions) for determining whether to associate a hose with itself or not. These include the following: (1) the end of hose 56 opposite patient temperature management device 46 must be positioned within a volume of space C; (2) the end of hose 56 opposite patient temperature management device 46 must be positioned within a threshold distance D of an associated thermal wrap 54; (3) the other end of the hose 56 must be positioned within volume of space A; and (4) the other end of the hose 56 must be positioned within the threshold distance B of a patient temperature management device 46. The four conditions are discussed in more detail below.

The first association condition of a hose 56 that must be met is for one end of the hose to be positioned within a volume of space C, one example of which is shown in FIG. 6. Volume of space C is smaller than volume of space A. This is because one end of the hose 56, when used with the patient on patient support apparatus 20, will be connected to a thermal wrap 54 that is worn by the patient. Accordingly, that end of the hose 56 will be positioned adjacent the patient when it is being used with that particular patient. Volume A is therefore chosen to only encompass the immediate area of patient support apparatus 20, such as, in some embodiments, the area above support deck 30. In general volume A is chosen such that it does not encompass any space in which a second patient support apparatus may be positioned, thereby excluding the possibility of incorrectly associating a hose 56 used on an adjacent patient support apparatus with patient support apparatus 20.

The second association condition of hose 56 (FIG. 8) requires that the end of the hose 56 positioned in volume of space C must also be positioned within a threshold distance D of an associated thermal wrap 54. Threshold distance D is automatically measured by communications between UWB transceiver 230 of thermal wrap 54 and the UWB transceiver 224 of hose 56 that is positioned on the end of hose 56 opposite patient temperature management device 46. In some embodiments, threshold distance D is the same as threshold distance B. However, in other embodiments, threshold distance D may be smaller or larger than threshold distance B. As with threshold B, however, threshold D is chosen so that it is generally not met if hose 56 does not have its connector 222 connected to one or more of the hose ports 232 of an associated thermal wrap 54. The exact value of threshold distance D may vary in accordance with the position of UWB transceiver 230 relative to hose ports 232 of the thermal wrap 54, and/or in accordance with the position of UWB transceiver 224 relative to connector 222. In general, the closer UWB transceiver 230 is positioned to one or more of ports 232, and the closer UWB transceiver 224 is positioned to connector 222, the smaller the value of threshold distance D, and vice versa.

The third association condition of hose 56 (FIG. 8) requires that the second end of hose 56 is positioned within volume of space A. Volume of space A was discussed above with respect to patient temperature management device 46 and need not be repeated herein other than to say that, if the second end of hose 56 is not positioned within volume of space A, controller 216 of patient support apparatus 20 will not associate the hose 56 with itself.

The fourth association condition of hose 56 (FIG. 8) is the same as that discussed above with respect to the association conditions for patient temperature management device 46; namely, the second end of hose 56 must be positioned within a threshold distance B of a patient temperature management device 46. This distance, as noted, is measured through UWB communications between UWB transceiver 224 positioned as the second end of hose 56 and the UWB transceiver 118 integrated into patient temperature management device 46.

As can be seen from the four association conditions of hose 56, one the conditions—that a first end of hose 56 is positioned within a threshold distance D of an associated wrap 54—requires that a thermal wrap 54 must first be associated with the patient support apparatus 20 before the hose 56 can be associated with the patient support apparatus 20. As a result, patient support apparatus 20 must first determine whether a thermal wrap 54 is associated with patient support apparatus 20 before it can ultimately decide on whether to associate hose 56 with itself or not.

As can be seen from FIG. 8, patient support apparatus 20 associates a thermal wrap 54 with itself if the thermal wrap 54 is positioned inside of volume of space C. An example of a volume of space C is shown in FIG. 6 and was previously discussed with respect to the association process of hose 56. As was noted, volume of space C is generally smaller than volume of space A and defined such that it does not encompass any space outside of patient support apparatus 20 that could potentially be occupied by another patient support apparatus and/or another patient. If a thermal wrap 54 is positioned inside of volume of space C, controller 216 associates that thermal wrap 54 with patient support apparatus 20.

As is shown in FIG. 8, controller 216 of patient support apparatus 20 may be further adapted to automatically associate other devices with patient support apparatus 20, such as, but not limited to, a display device 182, a vital sign sensor (not shown), and/or a fixed locator 142. In some embodiments, controller 216 of patient support apparatus 20 associates a display device 182 with patient support apparatus 20 if either of two different association conditions are met. First, if the display device 182 is positioned inside of a volume of space E, controller 216 associates the display device 182 with patient support apparatus 20. Second, if the display device 182 is coupled by a cable to a fixed locator 142 positioned inside of a space volume G.

Turning to the first manner of associating a display device 182 with patient support apparatus 20, space volume E is generally larger than space volume A, although the two may be of the same or similar size. Space volume E is generally defined to encompass the perimeter of patient support apparatus 20 in which a patient support apparatus 20 is positioned. Controller 216 determines if a display device 182 is positioned within volume of space E by utilizing UWB communications between transceivers 212 and at least one UWB transceiver (not shown) built into, or attached to, the display device 182.

Turning to the second manner of associating a display device 182 with patient support apparatus 20, controller 216 is configured to automatically associate a display device 182 with itself if the display device 182 is coupled by a display cable 180 to a fixed locator 142 that is positioned inside of a space volume G. Such a situation is illustrated in FIG. 7, which shows display device 182 coupled to fixed locator 142 by cable 180. Space volume G is chosen such that it will encompass both patient support apparatus 20 and fixed locator 142 when patient support apparatus 20 is positioned generally nearby to fixed locator 142 (e.g. within about one to three meters for linked locator units 142 and within potentially larger distances for unlinked locator units 142). In general, space volume G is dimensioned such that it will not encompass more than one locator unit 142 at any time. Additionally, it may be selected in accordance with the desired amount of granularity of the determination of the location of the patient support apparatus 20 (larger sizes for less granularity and smaller sizes for more granularity). Patient support apparatus 20 uses UWB communications between transceivers 212 and UWB transceiver 186 to determine the relative position of a locator unit 142 to patient support apparatus 20 (and thus whether or not it is positioned inside of space volume G).

Controller 216 of patient support apparatus 20 may also be configured to automatically associate one or more vital sign sensors with itself if those vital sign sensors are positioned within a volume of space F (FIG. 8). Volume of space F is chosen to encompass the space around patient support apparatus 20 in which a vital sign sensor may typically be positioned when used with the patient assigned to patient support apparatus 20. Volume of space F is also chosen to be small enough to prevent it from encompassing spaces in which a vital sign sensor used with another patient assigned to another, adjacent patient support apparatus may be positioned. Further details regarding the manner in which patient support apparatus 20 may automatically associate itself with one or more vital sign sensors, as well as the manner in which patient support apparatus 20 may process data from the one or more vital sign sensors, is disclosed in commonly assigned U.S. patent application Ser. No. 63/306,279 filed Feb. 3, 2022, by inventors Madhu Thota et al. and entitled COMMUNICATION SYSTEM FOR PATIENT SUPPORT APPARATUS, the complete disclosure of which is incorporated herein by reference. The aforementioned '279 application also discloses further details regarding the manner in which patient support apparatus 20 may automatically associate itself with a fixed locator 142, and regarding the communications between patient support apparatus 20 and the fixed locator 142. In some embodiments, patient support apparatus 20 may also or alternatively be configured to determine which locator units 142 (linked or unlinked) it is to associate itself with in any of the same manners that the patient support apparatuses and wall units carry out such association in commonly assigned U.S. patent application Ser. No. 63/245,245 filed Sep. 17, 2021, by inventors Kirby Neihouser et al. and entitled SYSTEM FOR LOCATING PATIENT SUPPORT APPARATUSES, the complete disclosure of which is incorporated herein by reference.

As has been described above, the decision as to whether to associate a patient support apparatus 20 with one or more devices (e.g. patient temperature management device 46, thermal wrap 54, hose 56, display device 182, vital sign sensor, and/or a fixed locator 142) is carried out by controller 216 of patient support apparatus 20. It will be understood, however, that this decision may alternatively be carried out by other structures. For example, in some embodiments, controller 216 is configured to send the spatial relationship data it determines from the communications between the various UWB transceivers (212, 186, 118, 224, and 230) to patient support apparatus server 138 and patient support apparatus server 138 then determines whether to associate any of these devices with patient support apparatus 20. In still other embodiments, one or more of the devices may include its own controller that determines whether it should be associated with patient support apparatus 20 and then forwards that information to patient support apparatus 20, to patient support apparatus server 138, to EMR server 136, and/or to another recipient.

It will also be understood that, although table 250 of FIG. 8 identifies a plurality of different volumes of space (e.g. volume of space A, C, E, F, and G), any two or more of these volumes of space may have the same size, the same position, and/or the same shape. In other words, although table 250 of FIG. 8 uses different letters to refer to these multiple volumes of space, this does not necessarily mean that the volumes of space are different in all embodiments. Still further, it will be understood that substantial modifications may be made to the association conditions shown in table 250. For example, in some embodiments, hose 56 may be modified to include only a single UWB transceiver 224 that is used for determining its association status and/or the association status of patient temperature management device 46. As another alternative, thermal wraps 54 may omit a UWB transceiver 230 and controller 216 may be modified to associate a hose 56 with patient support apparatus 20 if only three conditions are met instead of four: (1) a first end of the hose 56 is positioned within space volume C, (2) a second end of the hose 56 is positioned in volume of space A, and (3) the second end is within a threshold distance B to a patient temperature management device 46. Still other variations of the association conditions possible.

It will also be understood that the determination of the relative positions of the devices listed in table 250 and patient support apparatus 20 are carried out using UWB transceivers 212 and the UWB transceivers positioned aboard the various devices. For the measurement of the threshold distances B and D used with hoses 56, these distances may be determined by UWB communications between UWB transceivers 224, 118, and 230. Alternatively, or additionally, controller 216 may determine the position of each UWB transceiver 118, 224, and 230 using communications between these transceivers and the onboard UWB transceivers 212, and then use those position determinations to determine whether the threshold distances B and D are currently met.

Once an association has been made between a particular device and patient support apparatus 20, a further association may be made between that particular patient support apparatus 20 and a particular patient. This task of associating and disassociating a particular patient to a particular patient support apparatus 20 may also be carried out locally by controller 216, or it may be carried out remotely by patient support apparatus server 138. Such remote association to a particular patient generally involves patient support apparatus server 138 using information from ADT server 170 or EMR server 136 on network 134 to determine the room location (e.g. room number and/or bed bay ID) of a particular patient, and then matching that room location with the room location of a particular patient support apparatus 20 (which is reported to server 138 by the patient support apparatuses 20 which use locator units 142 to determine their location). In other words, server 138 consults a conventional server on network 134 that correlates specific patients to specific room numbers and/or bay areas, such as ADT server 170 and/or EMR server 136, and then uses the known room numbers and/or bay areas of specific patient support apparatuses 20 to match a specific patient to a specific patient support apparatus 20.

Local association of a particular patient support apparatus 20 and a particular patient may be carried out in a variety of different manners. One manner involves incorporating a bar code scanner and/or near field sensor into patient support apparatus 20 that is adapted to read the bar code and/or near field patient ID data contained within a conventional patient wristband. When a patient is assigned to a particular patient support apparatus 20, the caregiver scans the patient's wristband using the bar code or near field scanner that is built into the patient support apparatus 20. The patient ID data that is read from the wristband is then forwarded to server 138, which may distribute it to EMR server 136 and/or to other servers on the network 134. Controller 216 may also be adapted to include this patient ID data in the patient temperature management data it receives from patient temperature management device 46 and reports to server 138 (and/or to another server on network 134) so that the servers know which specific patient the patient temperature management data corresponds to.

In some embodiments, when patient support apparatus 20 and/or server 138 associate a device with a particular patient support apparatus 20 and/or with a specific patient, controller 216 and/or server 138 are configured to inform medical personnel (via electronic devices 162) that the device is associated with a particular patient support apparatus 20 and/or with a particular patient assigned to that patient support apparatus 20. Patient support apparatus 20 may therefore be configured to automatically forward patient temperature data, vital sign data, and/or other data from other medical devices to server 138 after the corresponding devices become associated with patient support apparatus 20. Such data may, in turn, be automatically forwarded by server 138 to EMR server 136 for entry into the corresponding patient's electronic medical record. Alternatively, or additionally, such data may be forwarded by server 138 to one or more electronic devices 162 associated with corresponding caregivers so that the caregivers for the patient assigned to that particular patient may be remotely informed of the data from the associated medical devices.

Once controller 216 or server 138 associates a medical device with a patient support apparatus 20, data from the associated medical devices can be automatically recorded in that patient's particular electronic medical record without requiring the caregiver to associate the medical devices with the patient and/or with the patient support apparatus 20 assigned to that patient. In other words, because controller 216 automatically determines what medical devices it is associated with, it is not necessary for a caregiver to take any manual steps to ensure that data from these medical devices is forwarded to the proper corresponding patient's electronic medical record because patient support apparatus 20, along with server 138, automatically determine the correct patient associated with that medical device. Further details regarding at least one manner in which this automatic patient determination may be made are found in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which is incorporated herein by reference.

From a review of table 250 of FIG. 8, it can be seen that the association of patient temperature management device 46 with patient support apparatus 20 is dependent upon the association of other devices, such as hose 56. Further, as can be seen in table 250, the association conditions for a hose 56 are dependent upon one of its ends being within a threshold distance of an associated thermal wrap 54. Accordingly, the association status of a hose 56 to patient support apparatus 20 is also dependent upon the association status of another device; namely, a thermal wrap 54. It can therefore be seen that patient temperature management device 46 will only be associated with patient support apparatus 20 if a corresponding hose 56 and thermal wrap 54 are associated with patient support apparatus 20. This stands in contrast to some of the other association conditions of table 250, such as those of a vital sign monitor and/or a fixed locator 142, which can be associated to patient support apparatus 20 without regard to the position and/or association status of other devices.

In some embodiments, one or more of the association conditions are modifiable by authorized personnel. Such modifications may take place directly on a patient support apparatus 20 by utilizing control panel 44a or they may take place through accessing patient support apparatus server 138. In either situation, authorized personnel can adjust the size, shape, and/or position of one or more of the volumes of space A, C, and E-G, as well as the number conditions and/or content of the association conditions for individual devices.

It will be understood that, although each volume of space A, C, and E-G has been discussed herein as being defined with respect to patient support apparatus 20, and thus movable as patient support apparatus 20 moves, this may be modified. For example, in some embodiments, volume of space G (FIG. 8) is defined with respect to a locator unit 142 and does not move as the patient support apparatus 20 moves. In some embodiments of patient support apparatus 20, some of the volumes of space A, C, and/or E-G are defined with respect to patient support apparatus 20 and other(s) are defined with respect to the medical device whose association status is being determined.

It will also be understood that in any of the embodiments of patient support apparatus 20 discussed herein, the size, shape, location, and/or other aspects of volumes of space A, C, and E-G may be changed by controller 216 when determining whether to disassociate a device, rather than to associate a device. In other words, once a device has been determined to be positioned inside of a particular volume of space (and the other association conditions are met, if any), controller 216 may increase the size of—and/or otherwise change one or more dimensions of—the volume of space when determining whether to disassociate the device. In this manner, the volumes of space A, C, and/or E-G may have a sort of hysteresis aspect wherein a device has to be positioned inside of a smaller space volume in order to be associated with another device, but thereafter can only be disassociated if it moves outside of a larger sized volume of space. In still other embodiments, the dimensions of one or more of the volumes of space A, C, and/or E-G are the same for both association and disassociation purposes.

As was noted previously, patient support apparatus 20 may be adapted to automatically associate itself with a variety of other types of medical devices besides those listed in FIG. 8. The association of those devices may involve a determination of whether those devices are positioned inside of one of the volumes of space A, C, and/or E-G identified in FIG. 8, or they may involve the determination of whether those devices are positioned inside of a different volume of space. In some embodiments, controller 216 is adapted to automatically determine whether to associate and disassociate itself with any one or more of the medical devices disclosed in commonly assigned U.S. patent application 63/154,677 filed Feb. 27, 2021, by inventors Celso Pereira et al. and entitled SYSTEM FOR DETERMINING PATIENT SUPPORT APPARATUS AND MEDICAL DEVICE LOCATION, the complete disclosure of which is incorporated herein by reference. Patient support apparatus 20 is configurable by a user to also, or alternatively, automatically display any of the data from these other types of medical devices on a display device 182 when these medical devices are associated with patient support apparatus 20 and one or more display devices 182 (or a connected locator unit 142) are also associated with patient support apparatus 20.

It should also be noted that the display of data from an associated patient temperature management device 46, as well as any data from any other associated medical device, is carried out by patient support apparatus 20 automatically. That is, for example, when a user has configured patient support apparatus 20 to display a patient's temperature on a display device 182, it automatically forwards the temperature data to the display device 182 after the corresponding association has been made (e.g. patient support apparatus 20 has been associated with a display device 182 or a locator unit 142 to which a display device 182 is coupled). Consequently, if a patient with a temperature sensors is wheeled into a bay area of a patient room that includes a fixed display device 182 coupled to a locator unit 142, controller 216 is configured to automatically start displaying the patient's temperature on that display device 182 as soon as it completes the association process with the corresponding locator unit 142. In this manner, the caregiver sees the patient's temperature displayed on the display device 182 within seconds after moving the patient support apparatus 20 into the bay area, and the caregiver doesn't need to connect any cables, press any buttons, or take any other actions, in order for the temperature data to be displayed on display device 182. Similarly, when the patient support apparatus 20 moves out of the bay area, the display of the patient's data on the display device 182 automatically terminates.

The automatic display of data on one or more display devices 182 is able to be carried out by controller 216 because it repetitively re-evaluates the association status of itself to any medical devices that are within communication range of UWB transceivers 212. When a display device 182 and medical device are associated, controller 216 automatically forwards selected data from the medical device to the display 182. It will be understood that the frequency at which controller 216 repetitively re-evaluates the association of patient support apparatus 20 with other medical devices may vary in different embodiments. In some embodiments, this occurs multiple times a second. In other embodiments, this occurs less frequently than once a second. In still other embodiments, the frequency at which controller 216 determines the relative position of a device to patient support apparatus 20 (and thereafter is association status) may vary according to the device, the current location of patient support apparatus 20, the movement state of patient support apparatus 20 (i.e. whether it is currently moving or stationary), the brake state of patient support apparatus 20 (i.e. whether the brake is on or off), and/or according to one or more other factors.

FIG. 9 illustrates one example of the type of data that may be displayed on a display device 182. As shown therein, the display device 182 is displaying a patient's blood pressure 260, the patient's heart rate 262, the patient's respiration rate 264, the patient's saturated oxygen level 266, the patient's weight 268, a patient target temperature 270 that is to be met through the use of patient temperature management device 46, a current patient temperature 272, an elapsed amount of time 274 that has passed since therapy using patient temperature management device 46 commenced, an amount of cooling power 276 that patient temperature management device 46 is currently using, and any alarms 278 that may have been detected and/or generated by patient temperature management device 46.

The patient's target temperature 270, current temperature 272, elapsed therapy time 274, cooling power 276, and alarms 278 are sent by patient temperature management device 46 to patient support apparatus 20 using UWB transceiver 118 (or another onboard transceiver), and patient support apparatus 20 then forwards this data to display device 182. The blood pressure 260, heart rate 262, respiration rate 265, and saturated oxygen levels 266 come from corresponding vital sign sensors that are associated with patient support apparatus 20. As with patient temperature management device 46, these vital sign sensors forward their information to patient support apparatus 20, which then forwards this data to display device 182. The weight 268 comes from the internal scale system onboard patient support apparatus 20.

Display device 182 is also showing the room number in which patient support apparatus 20 is currently located (FIG. 9). As was discussed previously, in some embodiments, controller 216 is configured to determine the current location of patient support apparatus 20 after it receives the unique ID 190 from a locator unit 142, forwards that unique ID 190 along with its own unique ID 184 (FIG. 7) to patient support apparatus server 138, and server 138 sends a return message back to the patient support apparatus 20 (via access points 132 and network transceiver 160) that informs the patient support apparatus 20 of the location within the healthcare facility corresponding to that particular patient support apparatus 20.

Patient support apparatus 20 may include one or more screens that are displayable on display 52 that allow a user to customize the data that is shown on display device 182. In other words, the user can configure patient support apparatus 20 to change the example of displayed data shown in FIG. 9. In addition, patient support apparatus 20 is configured to allow the user to select what data, if any, from the associated devices is displayed on onboard display 52. Patient support apparatus 20 may also allow the user to change the format and/or layout in which the selected data is to be displayed.

When patient support apparatus 20 associates itself with a particular linked locator unit 142, controller 216 selects that particular linked locator unit 142 to send the patient's voice signals to (and/or exit detection alerts to) for forwarding to nurse call system 152. It is also the linked locator unit 142 that controller 216 sends television commands to when a patient onboard patient support apparatus 20 activates one or more of the television controls 50l-50r. Similarly, it is the linked locator unit 142 that controller 216 sends light commands to when a patient onboard patient support apparatus 20 activates one or more or the reading or room light controls 50s or 50t. The linked locator unit 142 that patient support apparatus 20 associates itself with is also the locator unit 142 that patient support apparatus 20 will receive audio signals from and direct to its onboard speaker(s). Such audio signals may correspond to voice signals from a remotely positioned nurse that are forwarded to the corresponding communication outlet 144 by way of nurse call system 152, or such audio signals may correspond to television audio signals that are routed from television 150 to communication outlet 144 by way of the one or more conductors 158.

As was mentioned previously, in some embodiments, one or more of the UWB transceivers that are coupled to a particular device (e.g. UWB transceivers 118, 186, 224, and/or 230) may be constructed as a tag that is attached to that particular device. Examples of the manner in which such tags may be constructed are disclosed in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which has already been incorporated herein by reference. The use of such tags allows controller 216 to automatically determine the position of devices that don't have their own built-in UWB transceivers, but that instead have a UWB tag attached to them.

In some embodiments, one or more of the UWB transceivers disclosed herein may operate in the same manner as, and include any of the same functions as, the anchors and pseudo-anchors disclosed in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which has already been incorporated herein by reference.

In any of the embodiments disclosed herein, server 138 may be configured to additionally execute a caregiver assistance software application of the type described in the following commonly assigned patent applications: U.S. patent application Ser. No. 62/826,097, filed Mar. 29, 2019 by inventors Thomas Durlach et al. and entitled PATIENT CARE SYSTEM; U.S. patent application Ser. No. 16/832,760 filed Mar. 27, 2020, by inventors Thomas Durlach et al. and entitled PATIENT CARE SYSTEM; and/or PCT patent application serial number PCT/US2020/039587 filed Jun. 25, 2020, by inventors Thomas Durlach et al. and entitled CAREGIVER ASSISTANCE SYSTEM, the complete disclosures of which are all incorporated herein by reference. That is, server 138 may be configured to share with one or more electronic devices 162 any of the information shared with the electronic devices disclosed in these aforementioned patent applications. Thus, for example, server 138 may be configured to not only share the location of patient support apparatuses 20 (and any medical devices that may be associated with them) with electronic devices 162, but it may also forward to devices 162 temperature data from patient temperature management device 46, vital sign data, patient support apparatus status data (e.g. current siderail position, bed exit status, brake status, height status, scale data, etc.) and/or caregiver rounding data (e.g. when the last rounding was performed for a particular patient, when the next rounds are due, etc.).

It will also be understood that the number of UWB transceivers 212 on patient support apparatus 20 may vary. In some embodiments, patient support apparatus 20 includes three UWB transceivers 212 positioned at known locations on patient support apparatus 20 that are stored in memory 218. In other embodiments, four UWB transceivers 212 are included. In still other embodiments, a different number of UWB transceivers 212 may be included.

In some embodiments, linked locator units 142 and/or unlinked locator units 142b may include additional information stored therein that is shared with patient support apparatus 20 when patient support apparatus 20 becomes associated with the locator unit 142. Such additional information may include location information identifying the relative position of the locator unit 142 with respect to one or more other locator units 142 that are positioned nearby. Additionally or alternatively, the locator units 142 may include information regarding the thickness and/or materials of the wall to which it is attached, wherein such information provides an indication to the patient support apparatus 20 of the amount of attenuation that UWB signals will likely experience when traveling through that wall. Additionally or alternatively, the locator units 142 may include information identifying their general location within the healthcare facility (e.g. room 400, bay A of room 302, hallway X, maintenance area Y, radiology department, emergency department, etc.) and/or information identifying a more specific location of the locator units 142 within the healthcare facility (e.g. a set of X,Y,Z coordinates in a frame of reference that includes all, or a portion of, the healthcare facility; a height on the wall 154, a distance from one or more landmarks and/or architectural features within the healthcare facility, and/or other more specific information). In some embodiments, patient support apparatus 20 is adapted to utilize this information to determine its location within the healthcare facility and/or to determine whether it is positioned on the same side of a wall as a particular locator unit 142. In some embodiments, patient support apparatus 20 and/or locator units 142 include any of the same structures, functions, and/or features of any of the patient support apparatuses and/or wall units disclosed in commonly assigned U.S. patent application Ser. No. 63/245,245 filed Sep. 17, 2021, by inventors Kirby Neihouser et al. and entitled SYSTEM FOR LOCATING PATIENT SUPPORT APPARATUSES, the complete disclosure of which has already been incorporated herein by reference.

It will be understood that the communication of data from one or more associated devices to one or more of the displays 182, as described herein, takes place without routing the data through network 134 and without utilizing network transceiver 160. Instead, this data may be transmitted directly from one UWB transceiver to another, such as from UWB transceiver 118 of patient temperature management device 46 to one of the UWB transceivers 212 of patient support apparatus 20, and from patient support apparatus 20 to locator unit 142 using UWB transceivers 212 and 186. Alternatively, one or more of these devices that transmit, or forward, vital sign data may use Bluetooth communication, or another form of direct communication. By using such direct communication, patient support apparatus 20 reduces the traffic load that would otherwise be present on network 134.

In any of the embodiments discussed herein, patient support apparatuses 20 and/or locator units 142 may be configured to determine ranging information with respect to one or more devices that are positioned within range of its UWB transceivers 212 (and/or 186), such as one or more of the following: a patient temperature management device 46, a thermal wrap 54, a hose 56, and/or one or more other types of medical devices. Such ranging information may include not only the distance between patient support apparatus 20 (or locator unit 142) and these devices, but also the angular orientation of these devices with respect to patient support apparatus 20, locator unit 142, and/or with respect to a common reference (e.g. north). In some embodiments, this ranging information may be forwarded to patient support apparatus server 138 and used to determine whether to associate one or more devices with patient support apparatus 20. In some embodiments, server 138 may be configured to use ranging information from multiple patient support apparatuses 20 and/or from multiple locator units 142 to determine whether to associate one or more devices with a particular patient support apparatus 20. in some embodiments, the ranging information that is generated may be of the same type, and/or processed in the same manner, as the ranging information discussed in, and illustrated in FIGS. 16 and 17 of, commonly assigned PCT patent application serial number PCT/US2022/017616 filed Feb. 24, 2022, by applicant Stryker Corporation and entitled SYSTEM FOR DETERMINING PATIENT SUPPORT APPARATUS AND MEDICAL DEVICE LOCATION, the complete disclosure of which is incorporated herein by reference.

As has been described above, the decision as to whether to associate a patient support apparatus 20 with one or more devices (e.g. patient temperature management device 46, thermal wrap 54, hose 56, display device 182, vital sign sensor, and/or a fixed locator 142) is carried out by controller 216 of patient support apparatus 20. It will be understood, however, that this decision may alternatively be carried out by other structures. For example, in some embodiments, controller 216 is configured to send the spatial relationship data it determines from the communications between the various UWB transceivers (212, 186, 118, 224, and 230) to patient support apparatus server 138 and patient support apparatus server 138 then determines whether to associate any of these devices with patient support apparatus 20. In still other embodiments, one or more of the devices may include its own controller that determines whether it should be associated with patient support apparatus 20 and then forwards that information to patient support apparatus 20, to patient support apparatus server 138, to EMR server 136, and/or to another recipient.

Although the foregoing description has primarily focused on controller 216 of patient support apparatus 20 and/or patient support apparatus server 138 determining the location of, and association status of, a set of devices (e.g. patient temperature management device 46, thermal wrap 54, hose 56, display device 182, vital sign sensor, and/or a fixed locator 142), it will be understood that patient support apparatus 20 and/or server 138 may be configured to determine the location and association status of a plurality of other devices not specifically mentioned above. Such devices include, but are not limited to, any one or more of the following: exercise devices, heel care boots, IV stands and/or poles, infusion pumps, ventilators, DVT pumps, patient monitors (e.g. saturated oxygen (SpO₂) monitors, EKG monitors, vital sign monitors, etc.), patient positioning devices (e.g. wedges, turning devices, pumps), ambient sensors (e.g. air temperature, air flow, light, humidity, pressure, altitude, sound/noise), mattress 42, an incontinence pad or one or more sensors adapted to detect patient incontinence, a Holter device adapted to monitor and record a patient's heart signals, a patient ID tag or bracelet worn by the patient that identifies the patient, a caregiver tag or ID bracelet worn by a caregiver that identifies the caregiver, one or more pieces of furniture that a patient may be expected to use, and/or other types of devices. In general, the devices whose position and association status may be determined by patient support apparatus 20 and/or server 138 may include any devices that are used in a medical setting for treating, diagnosing, monitoring, and/or caring for a patient.

It will also be understood by those skilled in the art that the use of the term “transceiver” throughout this specification is not intended to be limited to devices in which a transmitter and receiver are necessarily within the same housing, or share some circuitry. Instead, the term “transceiver” is used broadly herein to refer to both structures in which circuitry is shared between the transmitter and receiver, and transmitter-receivers in which the transmitter and receiver do not share circuitry and/or a common housing. Thus, the term “transceiver” refers to any device having a transmitter component and a receiver component, regardless of whether the two components are a common entity, separate entities, or have some overlap in their structures.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

1. A patient support apparatus comprising:
a support surface adapted to support a patient;
a first transceiver coupled to a first location on the patient support apparatus;
a second transceiver coupled to a second location on the patient support apparatus;
a third transceiver coupled to a third location on the patient support apparatus; and
a controller adapted to use radio frequency (RF) communication between the first, second, and third transceivers and a first and second device to determine a first position of the first device relative to the patient support apparatus and a second position of a second device relative to the patient support apparatus, the controller further adapted to use both the first position and the second position to determine whether or not to associate the first device with the patient support apparatus.

2. The patient support apparatus of claim 1 wherein the controller is further adapted to perform a first comparison of the first position to a first volume of space and to perform a second comparison of the second position to a second volume of space, and to use both the first and second comparisons to determine whether or not to associate the first device with the patient support apparatus.

3. The patient support apparatus of claim 1 wherein the controller is further adapted to associate the first device with the patient support apparatus if the first device is positioned inside of a first volume of space and the second device is positioned inside of a second volume of space different from the first volume of space, the controller adapted to not associate the first device with the patient support apparatus if any of the following are true: (a) the first device is positioned outside of the first volume of space, (b) the second device is positioned outside of the second volume of space, or (c) both the first device is positioned outside of the first volume of space and the second device is positioned outside of the second volume of space.

4. The patient support apparatus of claim 2 wherein the first device is a temperature management device adapted to deliver temperature controlled fluid to the patient, and the second device is a thermal pad adapted to wrap around a portion of the patient and receive the temperature controlled fluid from the temperature management device.

5. The patient support apparatus of claim 4 wherein the first volume of space is larger than the second volume of space.

6. The patient support apparatus of claim 2 wherein both the first and second volumes of space are defined in a fixed manner with respect to the patient support apparatus.

7-11. (canceled)

12. The patient support apparatus of claim 3 wherein the controller is adapted to associate the first device with the patient support apparatus if the first device is positioned inside of the first volume of space and the second device is positioned inside of the second volume of space, and to not associate the first device with the patient support apparatus if any of the following are true: (a) the first device is positioned outside of the first volume of space, (b) the second device is positioned outside of the second volume of space, or (c) both the first device is positioned outside of the first volume of space and the second device is positioned outside of the second volume of space.

13-14. (canceled)

15. The patient support apparatus of claim 1 wherein the controller is further adapted to use radio frequency (RF) communication between the first, second, and third transceivers to determine a third position of a third device relative to the patient support apparatus, and the controller is further adapted to also use the third position to determine whether or not to associate the first device with the patient support apparatus.

16. The patient support apparatus of claim 15 wherein the first device is a temperature management device adapted to deliver temperature controlled fluid to the patient, the second device is a first tag attached to a first end of a hose fluidly coupled to the temperature management device, and the third device is a second tag attached to a second end of the hose.

17. The patient support apparatus of claim 15 wherein the first device is a temperature management device adapted to deliver temperature controlled fluid to the patient, the second device is a thermal pad adapted to wrap around a portion of the patient and receive the temperature controlled fluid from the temperature management device, and the third device is a hose fluidly coupled to the temperature management device and the thermal pad.

18-28. (canceled)

29. A patient support apparatus comprising:
a support surface adapted to support a patient;
a first transceiver coupled to a first location on the patient support apparatus;
a second transceiver coupled to a second location on the patient support apparatus;
a third transceiver coupled to a third location on the patient support apparatus; and
a controller adapted to use radio frequency (RF) communication between the first, second, and third transceivers and a first device to determine a first position of the first device relative to the patient support apparatus; to receive a first ID from the first device; to use the first ID to determine a first association condition; and to use the first position and the first association condition to determine whether or not to associate the first device with the patient support apparatus.

30. The patient support apparatus of claim 29 wherein the first association condition defines a first volume of space inside which the first device must be positioned in order for the controller to associate the first device with the patient support apparatus.

31. The patient support apparatus of claim 30 wherein the first association condition further defines a second volume of space inside of which a second device must be positioned in order for the controller to associate the first device with the patient support apparatus; wherein the first association condition further defines a second ID of the second device; and wherein the first association condition further defines a first spatial condition between the first device and a third device that must be satisfied in order for the controller to associate the first device with the patient support apparatus, and a second spatial condition between the second device and a fourth device that also must be satisfied in order for the controller to associate the first device with the patient support apparatus.

32-33. (canceled)

34. The patient support apparatus of claim 31 wherein the first device is a temperature management device adapted to deliver temperature controlled fluid to the patient, the second device is a thermal pad adapted to wrap around a portion of the patient and receive the temperature controlled fluid from the temperature management device, the third device is a first tag attached to a first end of a hose fluidly coupled to the temperature management device and the thermal pad, the fourth device is a second tag attached to a second end of the hose.

35. The patient support apparatus of claim 34 wherein the first spatial condition defines a first threshold distance between the first device and the third device, and the second spatial condition defines a second threshold distance between the second device and the fourth device.

36-51. (canceled)

52. A patient support apparatus comprising:
a support surface adapted to support a patient;
a first transceiver coupled to a first location on the patient support apparatus;
a second transceiver coupled to a second location on the patient support apparatus;
a third transceiver coupled to a third location on the patient support apparatus; and
a controller adapted to use radio frequency (RF) communication between the first, second, and third transceivers and a first and second device in order to determine a first position of the first device relative to the patient support apparatus and a second position of the second device relative to the patient support apparatus, the controller further adapted to receive a first ID from the first device and a second ID from the second device and to use the first and second IDs to determine whether or not the first device is adapted to control the second device.

53. The patient support apparatus of claim 52 wherein the first device is a temperature management device adapted to deliver temperature controlled fluid to the patient, and the second device is a thermal pad adapted to wrap around a portion of the patient and receive the temperature controlled fluid from the temperature management device.

54. The patient support apparatus of claim 52 wherein the controller is further adapted to use the first ID and the second ID to determine a first association condition and a second association condition, respectively, to use the first position and the first association condition to determine whether or not to associate the first device with the patient support apparatus, and to use the second association condition and the second position to determine whether or not to associate the second device with the patient support apparatus.

55. The patient support apparatus of claim 54 wherein the first association condition defines a first volume of space in which the first device must be positioned in order for the controller to associate the first device with the patient support apparatus, and the second association condition defines a second volume of space in which the second device must be position in order for the controller to associate the second device with the patient support apparatus.

56. The patient support apparatus of claim 54 wherein the first and second association conditions are stored in a memory onboard the patient support apparatus and the controller is adapted to select the first and second association conditions based on the first and second IDs.

57-69. (canceled)
